(19) United States
(12) Patent Application Publication
(10) Pub. No.: US 2026/0317767 A1
(43) Pub. Date: Oct. 15, 2026

(54) Methods and Reagents for Improved Oligonucleotide Synthesis

(71) Applicant: Arrowhead Pharmaceuticals, Inc., Pasadena, CA (US)

(72) Inventors: Agnieszka Glebocka, Madison, WI (US); Szymon Klossowski, Middleton, WI (US); Zach Trilling, Madison, WI (US)

(21) Appl. No.: 19/712,693

(22) Filed: Jun. 18, 2026

Related U.S. Application Data
(63) Continuation of application No. PCT/US24/61173, filed on Dec. 19, 2024.
(60) Provisional application No. 63/612,531, filed on Dec. 20, 2023.
(60) Provisional application No. 63/634,860, filed on Apr. 16, 2024.
(60) Provisional application No. 63/683,214, filed on Aug. 14, 2024.

(51) Int. Cl. C12N 15/113

(52) U.S. Cl. CPC C12N 15/113; C12N 2310/14; C12N 2310/312; C12N 2310/321; C12N 2310/322; C12N 2310/3233; C12N 2330/00

(57) ABSTRACT

Provided herein are improved reagents and methods for synthesizing RNAi agents (e.g., oligonucleotides) with increased yields and purity.

### RELATED APPLICATIONS

This application is a continuation of PCT Application PCT/US24/61173, filed Dec. 19, 2024, which claims the benefit of priority to U.S. Provisional Patent Application Ser. No. 63/612,531, filed on Dec. 20, 2023, U.S. Provisional Patent Application Ser. No. 63/634,860, filed on Apr. 16, 2024, and U.S. Provisional Patent Application Ser. No. 63/683,214, filed on Aug. 14, 2024, the contents of each of which are incorporated herein by reference in their entirety.

### FIELD OF THE INVENTION

The present disclosure relates to improved reagents and methods for synthesizing RNAi agents (e.g., oligonucleotides).

### SEQUENCE LISTING

This application contains a Sequence Listing (in compliance with Standard ST26), which has been submitted in xml format and is hereby incorporated by reference in its entirety. The xml sequence listing file is named 30742-WO_SeqListing.xml, created Dec. 16, 2024, and is 80,994 bytes in size.

### BACKGROUND

It is of great interest for the therapeutic, diagnostic, reagent, and biological assay industries to have processes and methods for synthesizing highly pure oligonucleotides of various nucleotide sequences and lengths, at sufficiently high yields and minimal cost. Current oligonucleotide syntheses generally involve starting with a nucleotide or other similar moiety that is to be positioned at the 3′ end of the final oligonucleotide, which is attached to a solid support—e.g., a polymer or glass resin—and then employing chemical steps to sequentially add nucleotides, by covalent attachment, to the 5′ end to produce the final oligonucleotide. At the end of the synthesis run, the synthesized oligonucleotide is cleaved from the solid support.

Although, each individual step of the synthesis (e.g., coupling or cleavage of the final oligonucleotide) generally proceeds in high average yields, even slight decreases in yield can greatly impact the overall yield of the final oligonucleotide product. Linkers connecting an RNAi agent (e.g., an oligonucleotide) to a solid support that comprise a disulfide bond can lead to lower yields by undesired cleavage of the labile disulfide bond.

Thus, there is a need for more efficient and scalable methods to synthesize highly pure RNAi agents (e.g., oligonucleotides), with various nucleotide sequences, in a timely manner and at sufficiently high yields necessary for large-scale production.

### SUMMARY

In general, the present disclosure features improved synthesis of RNAi agents, compositions that include such RNAi agents, and methods for inhibiting expression of a synthesizing the RNAi agents and compositions that include the RNAi agents described herein.

In some aspects, provided herein is a functionalized solid support for oligonucleotide synthesis comprising the structure:

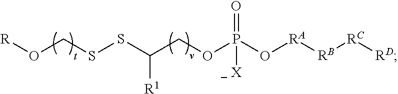

wherein R is hydrogen, a modified or unmodified nucleotide, a modified or unmodified oligonucleotide, an RNAi agent, or an oxygen protecting group; R¹ is substituted or unsubstituted C₁-C₆ alkyl; X is O or S; R^{A} is a modified or unmodified nucleotide; R^{B} is a linker; R^{C} is a spacer; R^{D} is a solid support material; t is an integer between 1 and 20, inclusive; and v is an integer between 1 and 20, inclusive.

In one aspect, provided herein is a functionalized solid support for oligonucleotide synthesis comprising the structure:

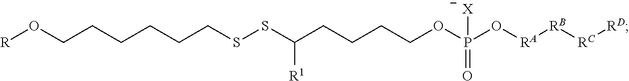

wherein R is hydrogen, a modified or unmodified nucleotide, a modified or unmodified oligonucleotide, an RNAi agent, or an oxygen protecting group; R¹ is substituted or unsubstituted C₁-C₆ alkyl; X is O or S; R^{A} is a modified or unmodified nucleotide; R^{B} is a linker; R^{C} is a spacer; and R^{D} is a solid support material.

In another aspect, provided herein is a compound of the formula (I):

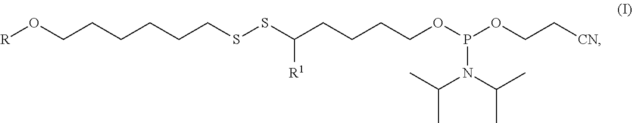

or a salt thereof, wherein:
R is hydrogen, a modified or unmodified nucleotide, or an oxygen protecting group; and R¹ is substituted or unsubstituted C₁-C₆ alkyl.

In some aspects, provided herein is a compound of the formula:

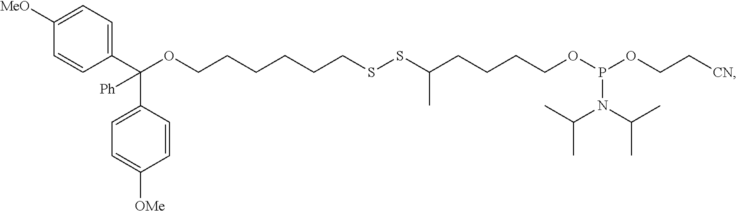 [SMILES: COc1ccc(C(OCCCCCCSSC(C)CCCCOP(OCCC#N)N(C(C)C)C(C)C)(c2ccccc2)c2ccc(OC)cc2)cc1]

or a salt thereof.

In other aspects, further provided herein is a compound of Formula (II):

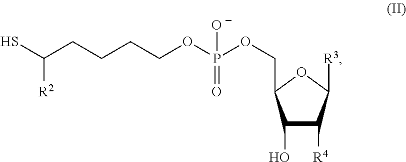

wherein:
R² is substituted or unsubstituted C₁-C₆ alkyl;
R³ is a nucleobase; and
R⁴ is hydrogen, methoxy, or a halogen.

In certain aspects, provided herein is a compound of the formula:

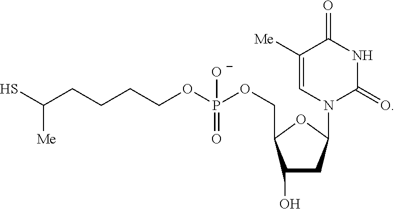

In one aspect, provided herein is a compound of the formula (III):

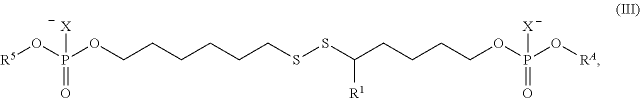

or a salt thereof, wherein R is hydrogen, a modified or unmodified nucleotide, an RNAi agent, or a modified or unmodified oligonucleotide; R^{A} is a modified or unmodified nucleotide; each X is independently O or S; and R¹ is substituted or unsubstituted C₁-C₆ alkyl.

In some aspects, provided herein is a compound of the formula:

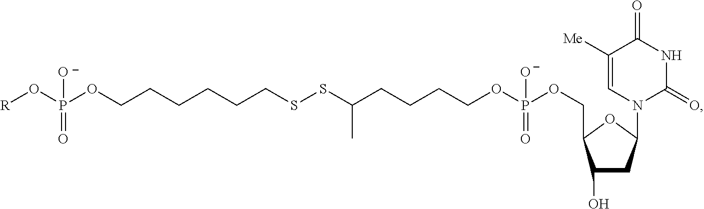

or a salt thereof, wherein R is a modified or unmodified nucleotide, an RNAi agent, or a modified or unmodified oligonucleotide.

In another aspect, further provided herein is a method of synthesizing an oligonucleotide comprising the step of reacting a support bound oligonucleotide comprising the structure:

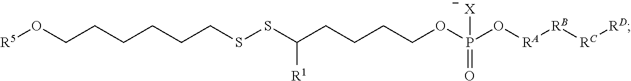

wherein R⁵ is a modified or unmodified oligonucleotide or an RNAi agent; R¹ is substituted or unsubstituted C₁-C₆ alkyl; R^{A} is a modified or unmodified nucleotide; R^{B} is a linker; R^{C} is a spacer; and R^{D} is a solid support material; with an organic base to form an oligonucleotide of the formula:

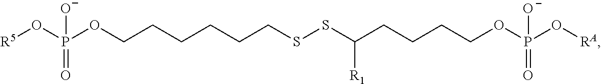

or a salt thereof.

In some aspects, provided herein is a method comprising the steps of reacting a compound of the formula:

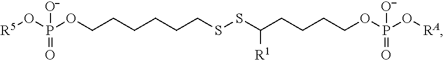

or a salt thereof, wherein R⁵ is a modified or unmodified oligonucleotide or an RNAi agent, R¹ is substituted or unsubstituted C₁-C₆ alkyl, and R^{A} is a modified or unmodified nucleotide; with a reducing agent to form a compound of the formula:

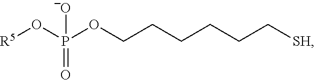

or a salt thereof.

In other aspects, provided herein is a method of synthesizing a compound of the formula:

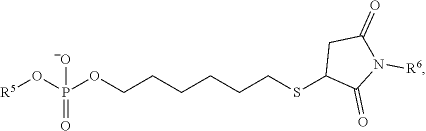

or a salt thereof, wherein R⁵ is a modified or unmodified oligonucleotide or an RNAi agent, and R⁶ is a PK/PD modulator, comprising the steps of:
(a) reacting a compound of the formula:

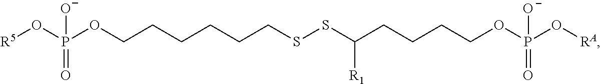

or a salt thereof wherein R⁵ is a modified or unmodified oligonucleotide or an RNAi agent, R¹ is substituted or unsubstituted C₁-C₆ alkyl, and R^{A} is a modified or unmodified nucleotide; with a reducing agent; and
(b) adding a compound of the formula:

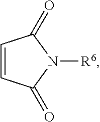

wherein R⁶ is a PK/PD modulator, to the reaction mixture.

In another aspect, further provided herein is a method of synthesizing a compound of the formula:

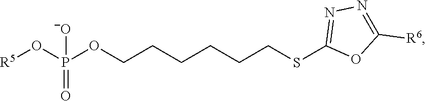

or a salt thereof, wherein R⁵ is a modified or unmodified oligonucleotide or an RNAi agent, and R⁶ is a PK/PD modulator, comprising the steps of:
(a) reacting a compound of the formula:

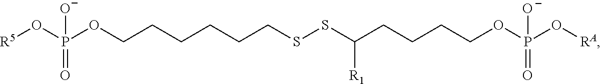

or a salt thereof wherein R⁵ is a modified or unmodified oligonucleotide or an RNAi agent, R¹ is substituted or unsubstituted C₁-C₆ alkyl, and R^{A} is a modified or unmodified nucleotide; with a reducing agent; and
(b) adding a compound of the formula:

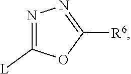

wherein L is a leaving group and R⁶ is a PK/PD modulator, to the reaction mixture.

Also provided herein is a method of synthesizing an oligonucleotide comprising the steps of:
(a) coupling a first nucleotide comprising a trityl group, or a first plurality of linked nucleotides comprising a trityl group to the functionalized solid support as described herein;
(b) optionally washing the functionalized solid support after coupling of the first nucleotide or the first plurality of linked nucleotides has been coupled to the solid support with a wash comprising acetic anhydride or N-methylimidazole (NMI), and an organic solvent;
(c) detritylating the first nucleotide or the first plurality of linked nucleotides with a reagent comprising a strong acid and an organic solvent to form a detritylated nucleotide or a plurality of detritylated linked nucleotides;
(d) washing the detritylated nucleotide or the plurality of detritylated linked nucleotides with a wash comprising a second organic solvent;
(e) coupling a second nucleotide comprising a trityl group, or a second plurality of linked nucleotides comprising a trityl group, to the detritylated nucleotide or the plurality of linked detritylated nucleotides under reaction conditions that promote coupling; and
(f) repeating steps (b) through (e) one or more times, wherein the method is carried out in a single reaction vessel.

### Definitions

For convenience, certain terms employed herein, in the specification, examples, and claims are collected herein.

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, the term “nucleotide” has the same meaning as commonly understood in the art, and thus refers to a glycoside comprising a sugar moiety, a base moiety and a covalently linked group (linkage group), such as a phosphate or phosphorothioate internucleoside linkage group, and covers both naturally occurring nucleotides, such as DNA or RNA, and non-naturally occurring nucleotides comprising modified sugar and/or base moieties, which are also referred to as nucleotide analogs or modified nucleotides herein. Herein, a single nucleotide can be referred to as a monomer or unit.

As used herein an “organic base” is an organic compound with basic properties (i.e., it can function as a proton acceptor/sink). Many, but not all, organic bases contain nitrogen atoms (e.g., amines). Non-limiting examples of organic bases include alkylamines (e.g., methylamine, triethylamine), amino acids (e.g., histidine, arginine, lysine), pyridine, and imidazole.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

Unless stated otherwise, use of the symbol

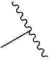

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Where a value is explicitly recited, it is to be understood that values which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Other objects, features, aspects, and advantages of the invention will be apparent from the following detailed description, accompanying figures, and from the claims.

### DETAILED DESCRIPTION

Described herein are improved methods and reagents for the synthesis of RNAi agents (e.g., oligonucleotides).

In certain embodiments, disclosed herein are functionalized solid supports for oligonucleotide synthesis comprising the structure:

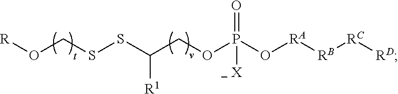

wherein R is hydrogen, a modified or unmodified nucleotide, a modified or unmodified oligonucleotide, an RNAi agent, or an oxygen protecting group;
R¹ is substituted or unsubstituted C₁-C₆ alkyl;
X is O or S;
R^{A} is a modified or unmodified nucleotide;
R^{B} is a linker;
R^{C} is a spacer;
R^{D} is a solid support material
t is an integer between 1 and 20, inclusive; and
v is an integer between 1 and 20, inclusive.

In certain embodiments, t is 0. In certain embodiments, t is 1. In certain embodiments, tis 2. In certain embodiments, t is 3. In certain embodiments, t is 4. In certain embodiments, t is 5. In certain embodiments, t is 6. In certain embodiments, t is 7. In certain embodiments, t is 8. In certain embodiments, t is 9. In certain embodiments, t is 10. In certain embodiments, t is 11. In certain embodiments, t is 12. In certain embodiments, t is 13. In certain embodiments, t is 14. In certain embodiments, t is 15. In certain embodiments, t is 16. In certain embodiments, t is 17. In certain embodiments, t is 18. In certain embodiments, t is 19. In certain embodiments, t is 20.

In certain embodiments, v is 0. In certain embodiments, v is 1. In certain embodiments, v is 2. In certain embodiments, v is 3. In certain embodiments, v is 4. In certain embodiments, v is 5. In certain embodiments, v is 6. In certain embodiments, v is 7. In certain embodiments, v is 8. In certain embodiments, v is 9. In certain embodiments, v is 10. In certain embodiments, v is 11. In certain embodiments, v is 12. In certain embodiments, v is 13. In certain embodiments, v is 14. In certain embodiments, v is 15. In certain embodiments, v is 16. In certain embodiments, v is 17. In certain embodiments, v is 18. In certain embodiments, v is 19. In certain embodiments, v is 20.

In certain embodiments, provided herein is a functionalized solid support for oligonucleotide synthesis comprising the structure:

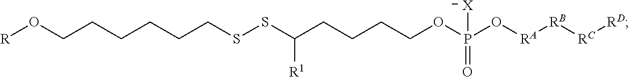

wherein R is hydrogen, a modified or unmodified nucleotide, a modified or unmodified oligonucleotide, an RNAi agent, or an oxygen protecting group;
R¹ is substituted or unsubstituted C₁-C₆ alkyl;
X is O or S;
R^{A} is a modified or unmodified nucleotide;
R^{B} is a linker;
R^{C} is a spacer; and
R^{D} is a solid support material.

In certain embodiments, the modified solid support described herein comprises moiety R^{A}. In certain embodiments, R^{A} is a modified nucleotide. In certain embodiments, R^{A} is an unmodified nucleotide. In certain embodiments, R^{A} is modified or unmodified adenosine nucleotide. In certain embodiments, R^{A} is modified or unmodified cytidine nucleotide. In certain embodiments, R^{A} is modified or unmodified guanosine nucleotide. In certain embodiments, R^{A} is modified or unmodified uridine nucleotide. In certain embodiments, R^{A} is modified or unmodified inosine nucleotide. In certain embodiments, R^{A} is a modified deoxythymidine nucleotide. In certain embodiments, R^{A} is an unmodified deoxythymidine nucleotide.

In certain embodiments, the modified solid support described herein comprises moiety X. In certain embodiments, X is O. In certain embodiments, X is S.

In certain embodiments, the solid support described herein comprises the structure:

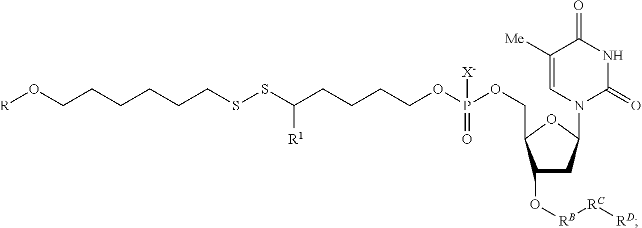

wherein R is hydrogen, a modified or unmodified nucleotide, an RNAi agent, or an oxygen protecting group;
R¹ is substituted or unsubstituted C₁-C₆ alkyl;
X is O or S;
R^{B} is a linker;
R^{C} is a spacer; and
R^{D} is a solid support material.

In certain embodiments, the solid supports described herein contain a linker group represented by moiety R^{B}. In certain embodiments, R^{B} is of the formula:

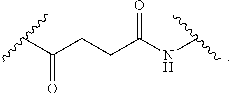 [SMILES: *NC(=O)CCC(*)=O]

In certain embodiments, the solid supports described herein comprise the structure:

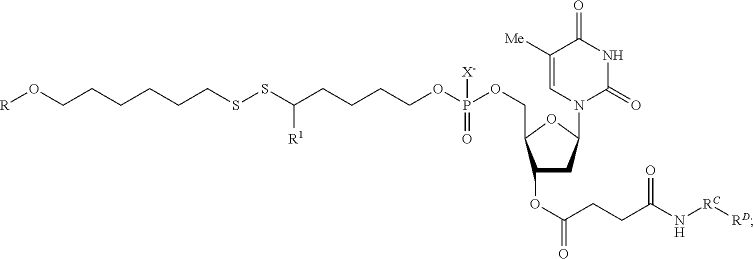

wherein R is hydrogen, a modified or unmodified nucleotide, an RNAi agent, or an oxygen protecting group;
R¹ is substituted or unsubstituted C₁-C₆ alkyl;
X is O or S;
R^{C} is a spacer; and
R^{D} is a solid support material.

In certain embodiments, the solid supports described herein contain a spacer group represented by moiety R^{C}. In certain embodiments, R^{C} is of the formula:

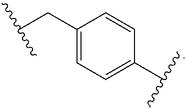 [SMILES: *Cc1ccc(*)cc1]

In certain embodiments, R^{C} is of the formula:

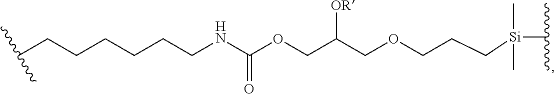

wherein R′ is C₁-C₆ alkyl.

In certain embodiments, the solid supports described herein comprise the structure:

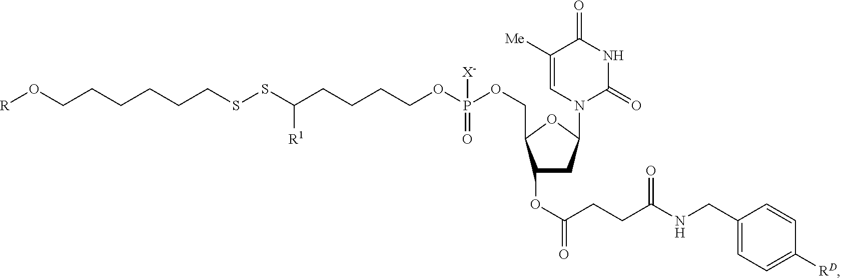

wherein R is hydrogen, a modified or unmodified nucleotide, a modified or unmodified oligonucleotide, an RNAi agent, or an oxygen protecting group;
X is O or S;
R¹ is substituted or unsubstituted C₁-C₆ alkyl; and
R^{D} is a solid support material.

In certain embodiments, the solid supports described herein comprise the structure:

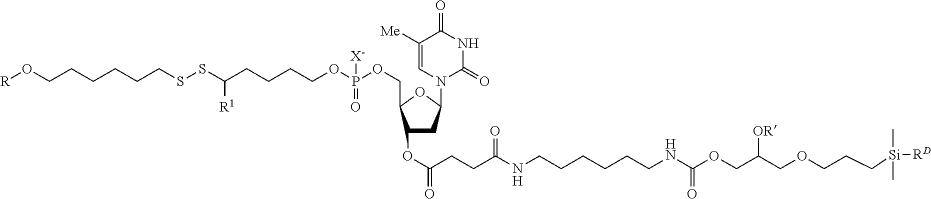

wherein R is hydrogen, a modified or unmodified nucleotide, a modified or unmodified oligonucleotide, an RNAi agent, or an oxygen protecting group;
X is O or S;
R¹ is substituted or unsubstituted C₁-C₆ alkyl;
R′ is substituted or unsubstituted C₁-C₆ alkyl; and
R^{D} is a solid support material.

In certain embodiments, the solid supports described herein comprise the moiety R¹. In certain embodiments, R¹ is substituted C₁-C₆ alkyl. In certain embodiments, R¹ is unsubstituted C₁-C₆ alkyl. In certain embodiments, R¹ is substituted or unsubstituted methyl. In certain embodiments, R¹ is unsubstituted methyl.

In certain embodiments, the solid supports described herein comprise the moiety R′. In certain embodiments, R′ is substituted C₁-C₆ alkyl. In certain embodiments, R′ is unsubstituted C₁-C₆ alkyl. In certain embodiments, R′ is substituted or unsubstituted methyl. In certain embodiments, R′ is unsubstituted methyl.

In some embodiments, the solid supports described herein comprise a solid support material represented the moiety R^{D}. In certain embodiments, the solid support material (i.e., R^{D}) is a commercially available solid support such as Nittophase™ (e.g., Nittophase™ HL) or Tentagel®. Tentagel® resins are grafted copolymers consisting of a low crosslinked polystyrene matrix on which poly(ethylene glycol) (PEG or POE) is grafted. Nittophase™ resins are cross-linked polystyrene polymer particles having a diameter of about 90 microns.

There are, however, other suitable solid phase materials known in the art that can be used with the present disclosure. Examples include but are not limited to a macroporous polystyrene polymer, a crosslinked polystyrene polymer, and a controlled pore glass. In some embodiments, the solid phase material comprises polymeric materials in form of particles with inner pores. In some embodiments, the solid phase material comprises glass in form of particles with inner pores. In some embodiments, the solid phase material comprises carbon in form of particles with inner pores.

In some embodiments, the solid phase material is made from one or more of polymers, resins, controlled porous glass, graphene, graphene oxide, and glass. In some embodiments, the solid phase material is of polymeric material. In some embodiments, the solid phase material is made of glass.

In certain embodiments, R^{D} comprises a polystyrene solid support. In certain embodiments, R^{D} comprises a controlled pore glass (CPG) solid support. In certain embodiments, R^{D} is a NittoPhase™ solid support. In certain embodiments, R^{D} is a NittoPhase™ HL solid support. In certain embodiments, R^{D} is a Primer Support™ solid support. In certain embodiments, R^{D} is a Primer Support™ 5G solid support.

In certain embodiments, the solid supports described herein contain the moiety R. In certain embodiments, R is an oxygen protecting group. In certain embodiments, R is a dimethoxytrityl (DMT) protecting group. In certain embodiments, R is a modified nucleotide. In certain embodiments, R is an unmodified nucleotide. In certain embodiments, R is an RNAi agent.

In certain embodiments, provided herein are compounds of the Formula (I′):

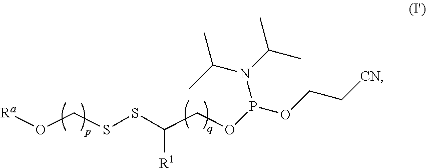

or a salt thereof, wherein:
R^{a} is hydrogen, a modified or unmodified nucleotide, an RNAi agent, or an oxygen protecting group;
R¹ is substituted or unsubstituted C₁-C₆ alkyl;
p is an integer between 0 and 20, inclusive; and
q is an integer between 0 and 20, inclusive.

In other embodiments, provided herein are compounds of the formula (V′):

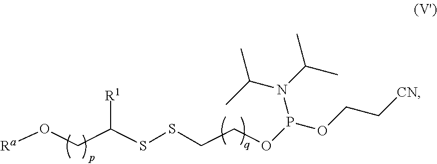

or a salt thereof, wherein:
R^{a} is hydrogen, a modified or unmodified nucleotide, an RNAi agent, or an oxygen protecting group;
R¹ is substituted or unsubstituted C₁-C₆ alkyl;
p is an integer between 0 and 20, inclusive; and
q is an integer between 0 and 20, inclusive.

In certain embodiments, p is 0. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8. In certain embodiments, p is 9. In certain embodiments, p is 10. In certain embodiments, p is 11. In certain embodiments, p is 12. In certain embodiments, p is 13. In certain embodiments, p is 14. In certain embodiments, p is 15. In certain embodiments, p is 16. In certain embodiments, p is 17. In certain embodiments, p is 18. In certain embodiments, p is 19. In certain embodiments, p is 20.

In certain embodiments, q is 0. In certain embodiments, q is 1. In certain embodiments, q is 2. In certain embodiments, q is 3. In certain embodiments, q is 4. In certain embodiments, q is 5. In certain embodiments, q is 6. In certain embodiments, q is 7. In certain embodiments, q is 8. In certain embodiments, q is 9. In certain embodiments, q is 10. In certain embodiments, q is 11. In certain embodiments, q is 12. In certain embodiments, q is 13. In certain embodiments, q is 14. In certain embodiments, q is 15. In certain embodiments, q is 16. In certain embodiments, q is 17. In certain embodiments, q is 18. In certain embodiments, q is 19. In certain embodiments, q is 20.

Also disclosed herein are compounds of the Formula (I):

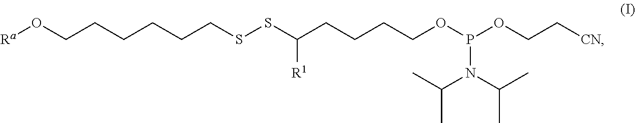

or a salt thereof, wherein:
R^{a} is hydrogen, a modified or unmodified nucleotide, an RNAi agent, or an oxygen protecting group; and R¹ is substituted or unsubstituted C₁-C₆ alkyl.

Further disclosed herein are compounds of Formula (V):

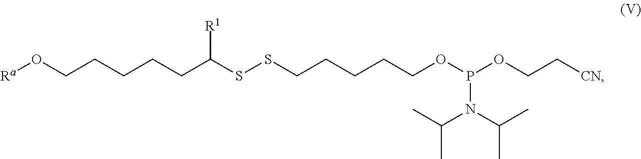

or a salt thereof, wherein:
R^{a} is hydrogen, a modified or unmodified nucleotide, an RNAi agent, or an oxygen protecting group; and R¹ is substituted or unsubstituted C₁-C₆ alkyl.

In certain embodiments, the compounds of Formula (I′), Formula (V′), Formula (I), and Formula (V) contain the substituent R¹. In certain embodiments, R¹ is unsubstituted C₁-C₆ alkyl. In certain embodiments, R¹ is substituted or unsubstituted methyl. In certain embodiments, R¹ is unsubstituted methyl.

In certain embodiments, the compounds of Formula (I′), Formula (V′), Formula (I), and Formula (V) contain the substituent R^{a}. In certain embodiments, R^{a} is hydrogen. In certain embodiments, R^{a} is a modified or unmodified nucleotide. In certain embodiments, R^{a} is an oxygen protecting group. In certain embodiments, R^{a} is a dimethoxytrityl (DMT) protecting group. In certain embodiments, R^{a} is an RNAi agent.

In certain embodiments, the compound of Formula (I′) or Formula (I) is of the formula:

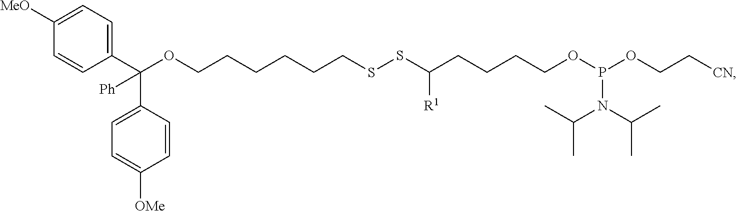

or a salt thereof.

In certain embodiments, the compound of Formula (I′) or Formula (I) is of the formula:

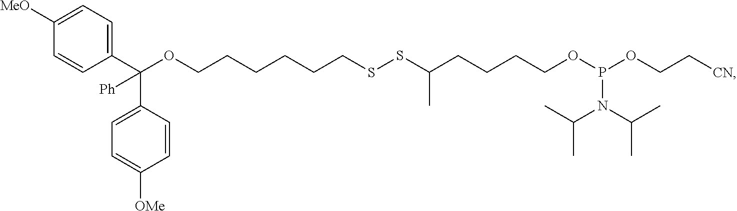 [SMILES: COc1ccc(C(OCCCCCCSSC(C)CCCCOP(OCCC#N)N(C(C)C)C(C)C)(c2ccccc2)c2ccc(OC)cc2)cc1]

or a salt thereof.

In certain embodiments, the compound of Formula (V′) or Formula (V) is of the formula:

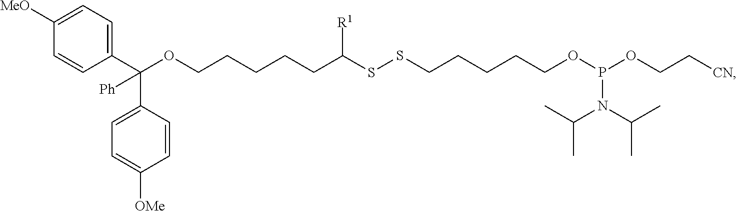

or a salt thereof.

In certain embodiments, the compound of Formula (V′) or Formula (V) is of the formula:

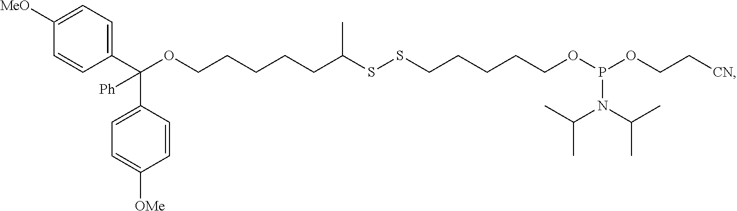 [SMILES: COc1ccc(C(OCCCCCC(C)SSCCCCCOP(OCCC#N)N(C(C)C)C(C)C)(c2ccccc2)c2ccc(OC)cc2)cc1]

or a salt thereof.

Also provided herein are compounds of Formula (II):

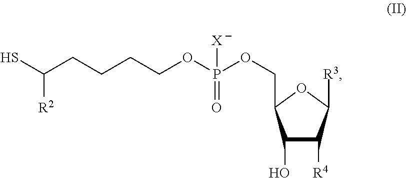

or a salt thereof, wherein:
R² is substituted or unsubstituted C₁-C₆ alkyl;
X is O or S;
R³ is a nucleobase; and
R⁴ is hydrogen, methoxy, or a halogen.

The compounds of Formula (II) as disclosed herein contain the substituent R². In certain embodiments, R² is substituted C₁-C₆ alkyl. In certain embodiments, R² is unsubstituted C₁-C₆ alkyl. In certain embodiments, R² is substituted or unsubstituted methyl. In certain embodiments, R² is unsubstituted methyl.

The compounds of Formula (II) as disclosed herein contain the substituent R³. In certain embodiments, R³ is cytosine. In certain embodiments, R³ is guanine. In certain embodiments, R³ is adenine. In certain embodiments, R³ is thymine. In certain embodiments, R³ is uracil.

The compounds of Formula (II) as disclosed herein contain the substituent R⁴. In certain embodiments, R⁴ is hydrogen. In certain embodiments, R⁴ is methoxy. In certain embodiments, R⁴ is a halogen.

In certain embodiments, the compound of Formula (II) is of the formula:

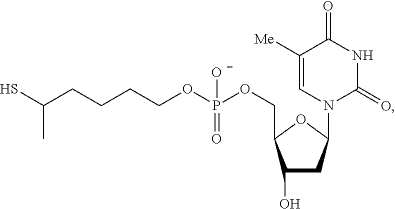

or a salt thereof.

In certain embodiments, provided herein are compounds of the Formula (III′):

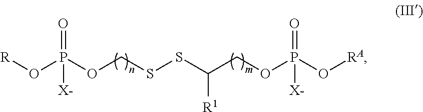

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S;
R¹ is substituted or unsubstituted C₁-C₆ alkyl;
n is an integer between 0 and 20, inclusive;
and m is an integer between 0 and 20, inclusive.

In other embodiments, provided herein are compounds of the Formula (IV′):

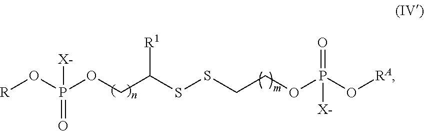

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S;
R¹ is substituted or unsubstituted C₁-C₆ alkyl;
n is an integer between 0 and 20, inclusive;
and m is an integer between 0 and 20, inclusive.

In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10. In certain embodiments, n is 11. In certain embodiments, n is 12. In certain embodiments, n is 13. In certain embodiments, n is 14. In certain embodiments, n is 15. In certain embodiments, n is 16. In certain embodiments, n is 17. In certain embodiments, n is 18. In certain embodiments, n is 19. In certain embodiments, n is 20.

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 13. In certain embodiments, m is 14. In certain embodiments, m is 15. In certain embodiments, m is 16. In certain embodiments, m is 17. In certain embodiments, m is 18. In certain embodiments, m is 19. In certain embodiments, m is 20.

Further provided herein are compounds of Formula (III):

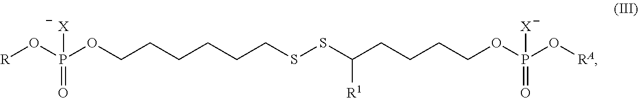

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S; and
R¹ is substituted or unsubstituted C₁-C₆ alkyl.

Further provided herein are compounds of the Formula (IV):

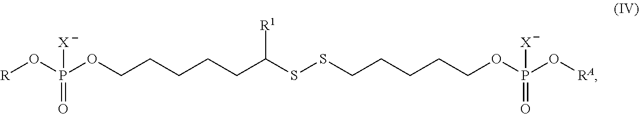

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S; and
R¹ is substituted or unsubstituted C₁-C₆ alkyl.

In certain embodiments, the compounds of Formula (III′), Formula (IV′), Formula (III), and Formula (IV) contain the substituent X. In certain embodiments, X is O. In certain embodiments, X is S.

In certain embodiments, the compounds of Formula (III′), Formula (IV′), Formula (III), and Formula (IV) contain the substituent R^{A}. In certain embodiments, R^{A} is a modified or unmodified nucleotide. In certain embodiments, R^{A} is modified or unmodified adenosine nucleotide. In certain embodiments, R^{A} is modified or unmodified cytidine nucleotide. In certain embodiments, R^{A} is modified or unmodified guanosine nucleotide. In certain embodiments, R^{A} is modified or unmodified uridine nucleotide. In certain embodiments, R^{A} is modified or unmodified inosine nucleotide. In certain embodiments, R^{A} is modified or unmodified deoxythymidine nucleotide.

In certain embodiments, the compounds of Formula (III′), Formula (IV′), Formula (III), and Formula (IV) contain the substituent R¹. In certain embodiments, R¹ is unsubstituted C₁-C₆ alkyl. In certain embodiments, R¹ is substituted or unsubstituted methyl. In certain embodiments, R¹ is unsubstituted methyl.

In certain embodiments, the compounds of Formula (III′) and Formula (III) are of the formula:

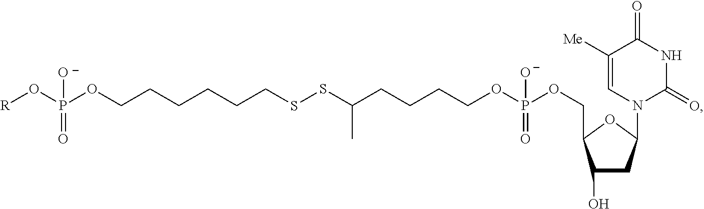

or a salt thereof, wherein R is hydrogen, a modified or unmodified nucleotide, an RNAi agent, or a modified or unmodified oligonucleotide.

In certain embodiments, the compounds of Formula (IV′) and Formula (IV) are of the formula:

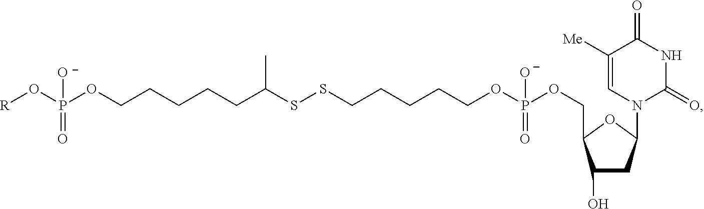

or a salt thereof, wherein R is hydrogen, a modified or unmodified nucleotide, an RNAi agent, or a modified or unmodified oligonucleotide.

In certain embodiments, the compounds of Formula (III′), Formula (IV′), Formula (III), and Formula (IV) as described herein contain the substituent R. In certain embodiments, R is hydrogen. In certain embodiments, R is an RNAi agent. In certain embodiments, R is a modified or unmodified nucleotide. In certain embodiments, R is a modified or unmodified oligonucleotide. In certain embodiments, the oligonucleotide comprises one or more modified nucleotides. In certain embodiments, the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide. In certain embodiments, the oligonucleotide comprises one or more modified internucleoside linkages. In certain embodiments, the oligonucleotide comprises a sequence that is between 18 and 30 nucleotides in length. In certain embodiments, the oligonucleotide comprises a sequence that is between 18 and 24 nucleotides in length. In certain embodiments, the oligonucleotide comprises a sequence that is 21 nucleotides in length.

Further provided herein is a method comprising the steps of reacting a compound of the formula:

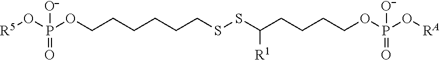

wherein R⁵ is a modified or unmodified oligonucleotide or an RNAi agent,
R¹ is substituted or unsubstituted C₁-C₆ alkyl, and
R^{A} is a modified or unmodified nucleotide;
with a reducing agent to form a compound of the formula:

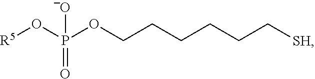

or a salt thereof.

In certain embodiments, the method further comprises step of reacting the compound of the formula:

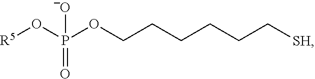

or a salt thereof wherein R⁵ is a modified or unmodified oligonucleotide, with a compound of the formula:

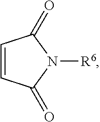

wherein R⁶ is a PK/PD modulator, to form a compound of the formula:

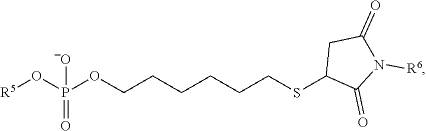

or a salt thereof.

In certain embodiments, the method further comprises the step of reacting the compound of the formula:

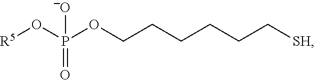

or a salt thereof, wherein R⁵ is a modified or unmodified oligonucleotide with a compound of the formula:

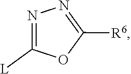

wherein
R⁶ is a PK/PD modulator, and
L is a leaving group;
to form a compound of the formula:

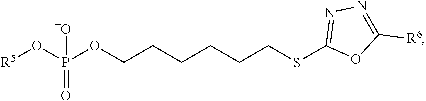

or a salt thereof.

Also provided herein is a method of synthesizing a compound of the formula:

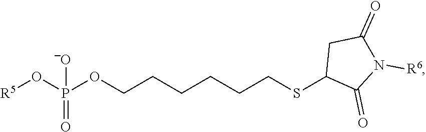

or a salt thereof, wherein:
R⁵ is a modified or unmodified oligonucleotide or an RNAi agent, and
R⁶ is a PK/PD modulator, comprising the steps of:
(a) reacting a compound of the formula:

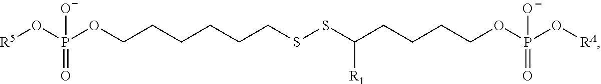

or a salt thereof
wherein R⁵ is a modified or unmodified oligonucleotide,
R¹ is substituted or unsubstituted C₁-C₆ alkyl, and
R^{A} is a modified or unmodified nucleotide or an RNAi agent;
with a reducing agent; and
(b) adding a compound of the formula:

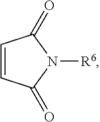

wherein R⁶ is a PK/PD modulator, to the reaction mixture.

Further provided herein is a method of synthesizing a compound of the formula:

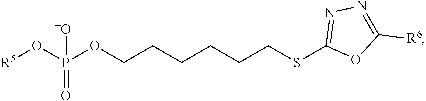

or a salt thereof, wherein:
R⁵ is a modified or unmodified oligonucleotide or an RNAi agent, and
R⁶ is a PK/PD modulator, comprising the steps of:
(a) reacting a compound of the formula:

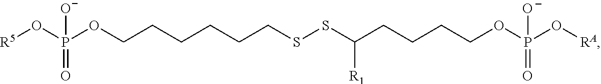

or a salt thereof
wherein R⁵ is a modified or unmodified oligonucleotide an RNAi agent,
R¹ is substituted or unsubstituted C₁-C₆ alkyl, and
R^{A} is a modified or unmodified nucleotide;
with a reducing agent; and
(b) adding a compound of the formula:

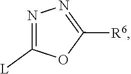

wherein L is a leaving group and R⁶ is a PK/PD modulator, to the reaction mixture.

In certain embodiments, L is a sulfonate leaving group. In certain embodiments, L is-SO₂Me.

In certain embodiments, the reducing agent is selected from the group consisting of tris (2-carboxyethyl) phosphine hydrochloride (TCEP), 2-mercaptoethanol, dithiothreitol (DTT), and hydroxylamine.

In certain embodiments, R^{A} is modified or unmodified adenosine nucleotide. In certain embodiments, R^{A} is modified or unmodified cytidine nucleotide. In certain embodiments, R^{A} is modified or unmodified guanosine nucleotide. In certain embodiments, R^{A} is modified or unmodified uridine nucleotide. In certain embodiments, R^{A} is modified or unmodified inosine nucleotide. In certain embodiments, R^{A} is modified or unmodified deoxythymidine nucleotide.

In certain embodiments, R¹ is substituted or unsubstituted methyl. In certain embodiments, R¹ is unsubstituted methyl.

In certain embodiments, R⁵ comprises one or more modified nucleotides. In certain embodiments, the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide. In certain embodiments, R⁵ comprises one or more modified internucleoside linkages. In certain embodiments, R⁵ comprises a sequence that is between 18 and 30 nucleotides in length. In certain embodiments, R⁵ comprises a sequence that is between 18 and 24 nucleotides in length. In certain embodiments, R⁵ comprises a sequence that is 21 nucleotides in length.

In certain embodiments, R⁶ is of the formula:

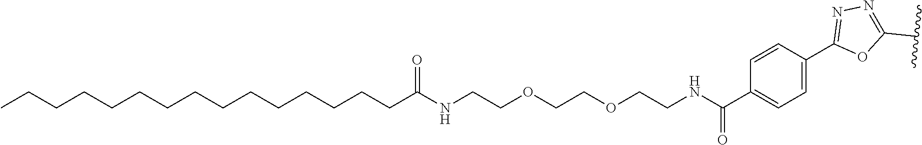 [SMILES: *c1nnc(-c2ccc(C(=O)NCCOCCOCCNC(=O)CCCCCCCCCCCCCCC)cc2)o1]

### Solid Phase Oligonucleotide Synthesis Methods

Further provided herein are methods of synthesizing an RNAi agent (e.g., an oligonucleotide) comprising the steps of reacting a support bound oligonucleotide comprising the structure:

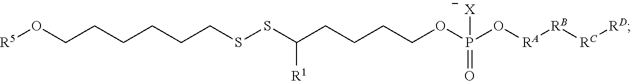

wherein R⁵ is a modified or unmodified oligonucleotide or an RNAi agent;
R¹ is substituted or unsubstituted C₁-C₆ alkyl;
R^{A} is a modified or unmodified nucleotide;
R^{B} is a linker;
R^{C} is a spacer; and
R^{D} is a solid support material;
with an organic base to form an oligonucleotide of the formula:

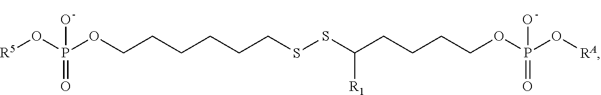

or a salt thereof.

In certain embodiments, R^{B} is of the formula:

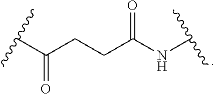 [SMILES: *NC(=O)CCC(*)=O]

In certain embodiments, R^{C} is of the formula:

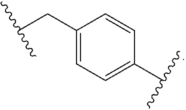 [SMILES: *Cc1ccc(*)cc1]

In certain embodiments, R^{C} is of the formula:

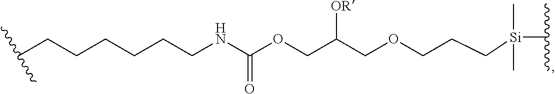

wherein R′ is C₁-C₆ alkyl.

In certain embodiments, the solid support material comprises a polystyrene solid support. In certain embodiments, the solid support is a controlled pore glass (CPG) solid support. In certain embodiments, the solid support is a commercially available solid support such as Nittophase™ (e.g., Nittophase™ HL).

In certain embodiments, the organic base is an amine base. In certain embodiments, the organic base is an alkylamine. In certain embodiments, the organic base is methylamine.

In certain embodiments, described herein is a method of synthesizing an oligonucleotide comprising the steps of:
(a) coupling a first nucleotide comprising a trityl group, or a first plurality of linked nucleotides comprising a trityl group to the functionalized solid support as described herein;
(b) optionally washing the functionalized solid support after coupling of the first nucleotide or the first plurality of linked nucleotides has been coupled to the solid support with a wash comprising acetic anhydride or N-methylimidazole (NMI), and an organic solvent;
(c) detritylating the first nucleotide or the first plurality of linked nucleotides with a reagent comprising a strong acid and an organic solvent to form a detritylated nucleotide or a plurality of detritylated linked nucleotides;
(d) washing the detritylated nucleotide or the plurality of detritylated linked nucleotides with a wash comprising a second organic solvent;
(e) coupling a second nucleotide comprising a trityl group, or a second plurality of linked nucleotides comprising a trityl group, to the detritylated nucleotide or the plurality of linked detritylated nucleotides under reaction conditions that promote coupling; and
(e) repeating steps (b) through (e) one or more times,
wherein the method is carried out in a single reaction vessel.

In various embodiments, the oligonucleotide assembly in the new methods occurs on the solid support via a series of step-wise reaction cycles, e.g., those listed below. Exemplary conditions for each reaction are further described in the Examples.

First, a trityl group of a support-bound nucleotide (e.g., a modified or unmodified thymidine) or a support-bound plurality of linked nucleotides is deblocked with a reagent comprising (i) an acid, and (ii) an organic solvent, liberating a 5′-hydroxyl group.

The second reaction involves coupling the appropriate nucleotide building block, in some embodiments a phosphoramidite ligand. In some embodiments the coupling step uses a coupling activator, such as 5-(Ethylthio)-1H-tetrazole (ETT), 4,5-dicyanoimidazole (DCI), N-methylimidazole (NMI), and/or 1H-tetrazole.

Third, the newly formed phosphite is then oxidized with an oxidation reagent, for example, a halogen/organic base/water solution (e.g., iodine/pyridine/water solution) to form a phosphate triester linkage. Other suitable organic bases include but are not limited to lutidine and collidine. Other suitable oxidation reagents include tert-butyl hydroperoxide (TBHP) and (1S)-(+)-(10-camphorsulfonyl)-oxaziridine (CSO), which may be used in anhydrous conditions. Alternatively, the newly formed phosphite is thiolated with thiolation reagent, for example, xanthane hydride.

Fourth, any unreacted 5′-OH groups are capped with a capping reagent. In some embodiments, the capping reagent comprises a first capping reagent, such as a solution of N-methylimidazole (NMI) in acetonitrile, and a second capping reagent, such as a solution of acetic anhydride (Ac2O), collidine, and acetonitrile. In other embodiments, a suitable capping reagent contains hydroxy protecting groups, such as esters and ethers. Non-limiting examples of suitable ethers and esters include methoxymethyl or MOM ether, tetrahydropyranyl or THP ether, tert-butyl ether, allyl ether, benzyl ether, tert-butyldimethylsilyl or TBDMS ether, tert-butyldiphenylsilyl or TBDPS ether, acetic acid ester, pivalic acid ester, and benzoate ester. In one embodiment, the unreacted 5′-OH groups are capped with acetic anhydride.

In various embodiments, at the start of a new cycle, the 5′-O-DMT-group of the coupled product is then deblocked, followed by addition of another nucleotide to the chain, and this process is repeated until an oligomer of the desired length and composition is obtained.

In various embodiments, by-products and excess reagents in solution are removed by filtration and washing the support with a wash solution, for example, acetonitrile solvent after some or all of the reaction steps.

The crude composition may be analyzed and confirmed, e.g., by Liquid Chromatography Mass Spectrometry (LCMS) before final purification.

The methods disclosed herein can produce yields of greater than about 90%, e.g., greater than 95%, or greater than 98% per cycle, and a yield of about 70% or greater for the final synthetic oligonucleotide composition. For instance, a method may produce a total yield of at least 0.5 mmol oligonucleotide after a single reactor run, optionally at least 20 mmol, optionally at least 60 mmol, optionally at least 1 mol, optionally at least 1.5 mol, optionally at least 2 mol.

The methods disclosed herein can have at least a 90% coupling efficiency, optionally a 95% coupling efficiency, optionally a 99% coupling efficiency.

Once the desired oligonucleotide has been synthesized on the solid support, it is then cleaved from the solid support to provide the desired oligonucleotide in solution.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administering of the oligonucleotide construct.

In some embodiments, an RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-methoxy (2′ internucleoside linked) nucleotides, 2′-F-Arabino nucleotides, 5′-Methyl, 2′-fluoro nucleotides, morpholino nucleotides, vinyl phosphonate-containing nucleotides, and cyclopropyl phosphonate-containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred to herein as 2′-deoxy-2′-fluoro nucleotides), 2′-deoxy nucleotides, 2′-methoxyethyl(2′-O-(2-methoxylethyl)) nucleotides (also referred to as 2′-MOE), 2′-amino nucleotides, and 2′-alkyl nucleotides. It is not necessary for all positions in a given compound to be uniformly modified. Conversely, more than one modification can be incorporated in a single RNAi agent or even in a single nucleotide thereof. Modification at one nucleotide is independent of modification at another nucleotide. Various modified nucleotides are well known and described in the art.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine (hypoxanthine), xanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some embodiments, one or more nucleotides of an RNAi agent is an unmodified ribonucleotide. Chemical structures for certain modified nucleotides are set forth in Table 1 herein.

### Modified Internucleoside Linkages

In some embodiments, one or more nucleotides of an RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, diphosphorothioates, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an RNAi agent can contain 1, 2, 3, 4, 5, or 6 phosphorothioate linkages, an antisense strand of an RNAi agent can contain 1, 2, 3, 4, 5, or 6 phosphorothioate linkages, or both the sense strand and the antisense strand independently can contain 1, 2, 3, 4, 5, or 6 phosphorothioate linkages.

In some embodiments, an RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 1). (See, e.g., F. Czauderna, Nucleic Acids Res., 2003, 31 (11), 2705-16; U.S. Pat. No. 5,998,203). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, one or more inverted abasic residues or inverted abasic sites are inserted between the PK/PD modulator and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb) s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. Chemical structures for inverted abasic deoxyribose residues are shown in Table 1 below.

The following notations are used herein to indicate modified nucleotides, targeting groups, and linking groups:

| | |
|---|---|
| A = | adenosine-3′-phosphate |
| C = | cytidine-3′-phosphate |
| G = | guanosine-3′-phosphate |
| U = | uridine-3′-phosphate |
| I = | inosine-3′-phosphate |
| a = | 2′-O-methyladenosine-3′-phosphate |
| as = | 2′-O-methyladenosine-3′-phosphorothioate |
| c = | 2′-O-methylcytidine-3′-phosphate |
| cs = | 2′-O-methylcytidine-3′-phosphorothioate |
| g = | 2′-O-methylguanosine-3′-phosphate |
| gs = | 2′-O-methylguanosine-3′-phosphorothioate |
| i = | 2′-O-methylinosine-3′-phosphate |
| is = | 2′-O-methylinosine-3′-phosphorothioate |
| t = | 2′-O-methyl-5-methyluridine-3′-phosphate |
| ts = | 2′-O-methyl-5-methyluridine-3′-phosphorothioate |
| u = | 2′-O-methyluridine-3′-phosphate |
| us = | 2′-O-methyluridine-3′-phosphorothioate |
| Af = | 2′-fluoroadenosine-3′-phosphate |
| Afs = | 2′-fluoroadenosine-3′-phosporothioate |
| Cf = | 2′-fluorocytidine-3′-phosphate |
| Cfs = | 2′-fluorocytidine-3′-phosphorothioate |
| Gf = | 2′-fluoroguanosine-3′-phosphate |
| Gfs = | 2′-fluoroguanosine-3′-phosphorothioate |
| Uf = | 2′-fluorouridine-3′-phosphate |
| Ufs = | 2′-fluorouridine-3′-phosphorothioate |
| dT = | 2′-deoxythymidine-3′-phosphate |
| a_2N = | see Table 1 |
| a_2Ns = | see Table 1 |
| (invAb) = | inverted abasic deoxyribonucleotide-5′- |
| | phosphate, see Table 1 |
| (invAb)s = | inverted abasic deoxyribonucleotide-5′- |
| | phosphorothioate, see Table 1 |
| s = | phosphorothioate linkage |
| p = | terminal phosphate (as synthesized) |
| cPrpa = | 5′-cyclopropyl phosphonate-2′-O-methyladenosine- |
| | 3′-phosphate (see Table 1) |
| cPrpas = | 5′-cyclopropyl phosphonate-2′-O-methyladenosine- |
| | 3′-phosphorothioate (see Table 1) |
| cPrpu = | 5′-cyclopropyl phosphonate-2′-O-methyluridine-3′- |
| | phosphate (see Table 1) |
| cPrpus = | 5′-cyclopropyl phosphonate-2′-O-methyluridine-3′- |
| | phosphorothioate (see Table 1) |
| (Alk-SS-C6) = | see Table 1 |
| (C6-SS-C6) = | see Table 1 |
| (C6-SS-Alk-Me) = | see Table 1 |
| (NH2—C6) = | see Table 1 |

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the RNAi agents described herein.

Certain examples of targeting groups and linking groups used with RNAi agents disclosed herein are included in the chemical structures provided below in Table 1. Each sense strand and/or antisense strand can have any targeting groups or linking groups listed herein, as well as other targeting or linking groups, conjugated to the 5′ and/or 3′ end of the sequence.

As discussed herein, in some embodiments, one or more targeting ligands and/or PK/PD modulators may be linked or conjugated to the RNAi agent. In some embodiments, a targeting ligand (or targeting group) and/or a PK/PD modulator is linked to the 5′ end of the sense strand, the 3′ end of the sense strand, and/or to one or more internal nucleotides. The synthesis of the sense strand and/or the antisense strand can be designed such that reactive groups are readily available to facilitate linkage to additional components, such as a targeting ligand or PK/PD modulator.

As described herein, in some embodiments, the duplexed sense strand nucleotide sequence and antisense strand nucleotide sequence can be linked to certain targeting ligands and/or PK/PD modulators.

In some embodiments, the synthesized RNAi agent is prepared or provided as a salt, mixed salt, a free-acid, or a free base. In some embodiments, an RNAi agent is prepared as a pharmaceutically acceptable salt. In some embodiments, a RNAi agent is prepared as a pharmaceutically acceptable sodium salt. Such forms that are well known in the art are within the scope of the inventions disclosed herein.

### Targeting Groups, Linking Groups, PK/PD Modulators, and Delivery Vehicles

In some embodiments, an RNAi agent as disclosed herein contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, a RNAi agent contains a non-nucleotide group linked to the 3′ and/or 5′ end of the sense strand. In some embodiments, a non-nucleotide group is linked to the 5′ end of a RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting ligands enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

The RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the RNAi agents disclosed herein are synthesized having an NH₂—C6 group (represented as (NH₂—C6) in the modified sequences herein) at the 5′-terminus of the sense strand of the RNAi agent. The terminal amino group subsequently can be reacted to form a conjugate with, for example, a group that includes a targeting ligand. In some embodiments, the RNAi agents disclosed herein are synthesized having one or more alkyne groups at the 5′-terminus of the sense strand of the RNAi agent. The terminal alkyne group(s) can subsequently be reacted to form a conjugate with, for example, a group that includes a targeting ligand.

In some embodiments, RNAi agents comprise a targeting group, which includes 2 or more targeting ligands. In some embodiments, a targeting group may be conjugated at the 5′ or 3′ end of the sense strand of an RNAi agent. In some embodiments, a targeting group may be conjugated to an internal nucleotide on an RNAi agent. In some embodiments, a targeting group may consist of two targeting ligands linked together, referred to as a “bidentate” targeting group. In some embodiments, a targeting group may consist of three targeting ligands linked together, referred to as a “tridentate” targeting group. In some embodiments, a targeting group may consist of four targeting ligands linked together, referred to as a “tetradentate” targeting group.

In some embodiments, the use of a targeting ligand facilitates cell-specific targeting to cells having desired receptors on its respective surface, and binding of the targeting ligand can facilitate entry of the therapeutic agent, such as an RNAi agent, to which it is linked, into cells. Targeting ligands can be monomeric or monovalent (e.g., having a single targeting moiety) or multimeric or multivalent (e.g., having multiple targeting moieties). The targeting group can be attached to the 3′ and/or 5′ end of the RNAi oligonucleotide using methods known in the art.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: C6-SS-C6, C6-SS-MeC5, 6-SS-6, reactive groups such as primary amines (e.g., NH2-C6) and alkynes, alkyl groups, abasic residues/nucleotides, amino acids, tri-alkyne functionalized groups, ribitol, and/or PEG groups.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, targeting groups are linked to the RNAi agents without the use of an additional linker. In some embodiments, the targeting group is designed having a linker readily present to facilitate the linkage to a RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, the RNAi agents described herein are linked to one or more lipid PK/PD moieties (referred to herein as “lipid moieties” or “PK/PD modulators”.) Lipid PK/PD moieties may enhance the pharmacodynamic or pharmacokinetic properties of the RNAi agent. In some embodiments, the lipid moiety may be conjugated to a linker at the 3′ or 5′ end of a sense strand or an antisense strand of an RNAi agent described herein. In some embodiments, a lipid moiety may be linked at both the 3′ or 5′ end of either the sense strand or the antisense strand of an RNAi agent described herein.

In some embodiments, a lipid moiety may be conjugated to an RNAi agent by reacting an RNAi agent comprising an amine-comprising linker, for example, (NH2-C6) (see table 1). In some embodiments, the amine-comprising linker may be located on the 5′ end of the sense strand or the antisense strand of an RNAi agent. In some embodiments, the amine-comprising linker may be located on the 3′ end of the sense strand or the antisense strand of an RNAi agent.

In some embodiments, an RNAi agent comprising an amine-comprising linker, such as (NH2-C6) or (NH2-C6) s, may be reacted with a lipid comprising an activated ester moiety.

In some embodiments, an RNAi agent comprising a disulfide linker, such as C6-SS-C6 or C6-SS-MeC5, may be reacted with a lipid comprising an aromatic sulfone, such as LP-371-p.

In some embodiments, an RNAi agent comprising a disulfide linker, such as C6-SS-C6 or C6-SS-MeC5, may be reacted with a lipid comprising a maleimide, such as LP-371-p.

In some embodiments, an RNAi agent comprising an alkyne linker, such as L6-p, may be reacted with a lipid comprising an azide, such as LP-379-p. The lipid may be reacted with L6 to form a triazole before or after an amidation reaction with an amine such as NH2-C6.

In some embodiments, RNAi agents may comprise a lipid moiety on an internal nucleotide (i.e., not on the 3′ or 5′ terminal nucleotides.) In some embodiments, a lipid moiety on an internal nucleotide may be linked to the 2′ position of ribose.

In certain embodiments, any of the RNAi agents described herein, whether modified or unmodified, can contain 3′ and/or 5′ targeting group(s), linking group(s), and/or lipid PK/PD moieties. In certain embodiments, any of the RNAi agent sequences described herein, which contain a 3′ or 5′ targeting group, linking group, and/or lipid PK/PD moiety can alternatively contain no 3′ or 5′ targeting group, linking group, or lipid PK/PD moiety, or can contain a different 3′ or 5′ targeting group, linking group, or lipid PK/PD moiety including, but not limited to, those depicted in Table 1.

Examples of certain modified nucleotides, capping moieties, lipid moieties, and linking groups are provided in Table 1.

TABLE 1
Structures Representing Various Modified Nucleotides and Linking Groups

| 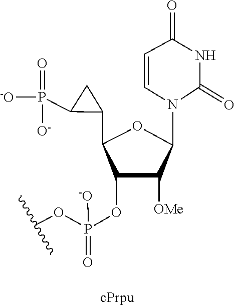 | 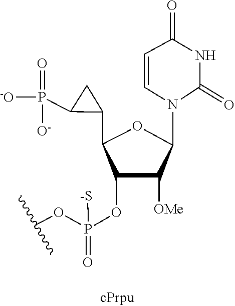 |
|---|---|
| 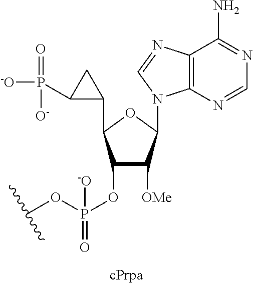 | 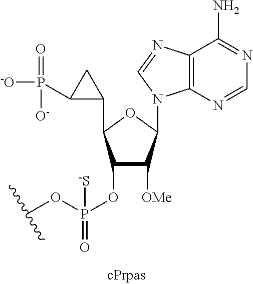 |
| 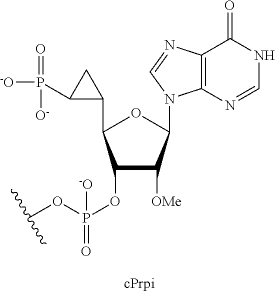 | 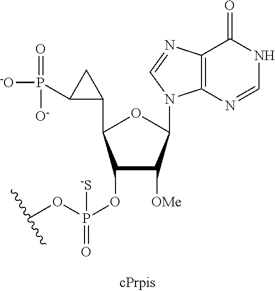 |
| 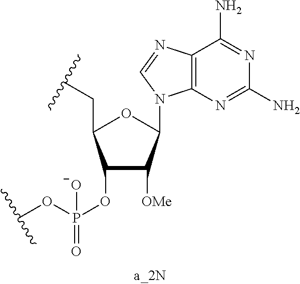 | 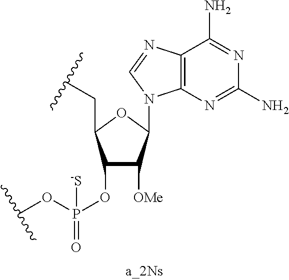 |
| When positioned internally: | |
| linkage toward 5′ end | linkage toward 5′ end |
| 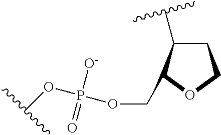 | 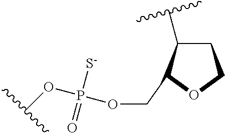 |
| linkage toward 3′ end | linkage toward 3′ end |
| (invAB) | (invAB)s |
| When positioned at the 3' terminal end: | |
| linkage toward 5′ end | |
| 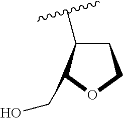 | |
| (invAB) | |
| When position at the 3' terminal end: | |
| linkage towards 5' end | |
| 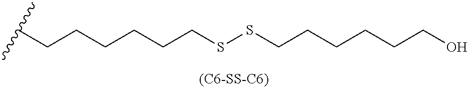 [SMILES: *CCCCCCSSCCCCCCO] | |
| When positioned internally: | |
| linkage towards 5' end linkage towards 3' end | |
| 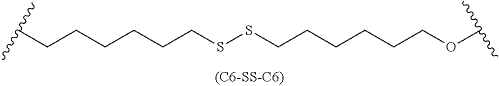 [SMILES: *CCCCCCSSCCCCCCO*] | |
| When position at the 3' terminal end: | |
| linkage towards 5' end | |
| 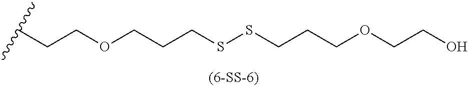 [SMILES: *CCOCCCSSCCCOCCO] | |
| When positioned internally: | |
| linkage towards 5' end linkage towards 3' end | |
| 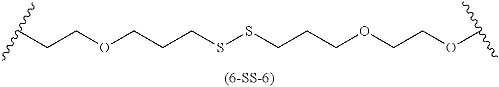 [SMILES: *CCOCCCSSCCCOCCO*] | |
| 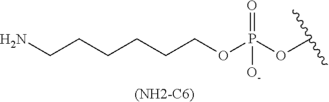 | 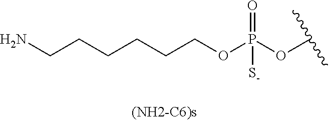 |
| 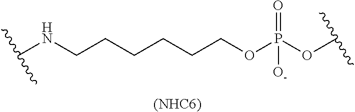 | 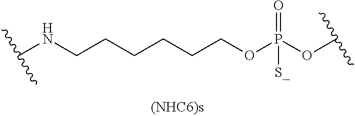 |

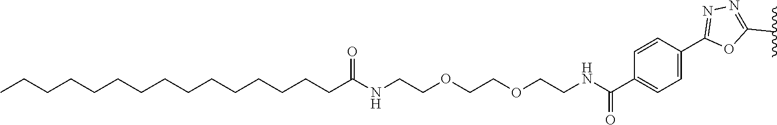 [SMILES: *c1nnc(-c2ccc(C(=O)NCCOCCOCCNC(=O)CCCCCCCCCCCCCCC)cc2)o1]

LP-371-a

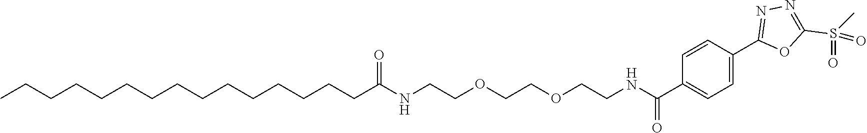 [SMILES: CCCCCCCCCCCCCCCC(=O)NCCOCCOCCNC(=O)c1ccc(-c2nnc(S(C)(=O)=O)o2)cc1]

LP-371-p

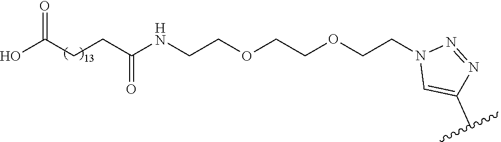

LP-379-a

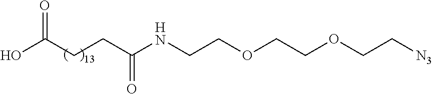

LP-379-p

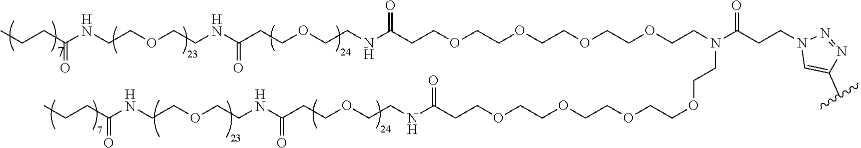

LP-238-a

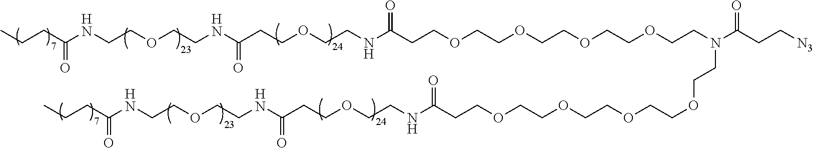

LP-238-p

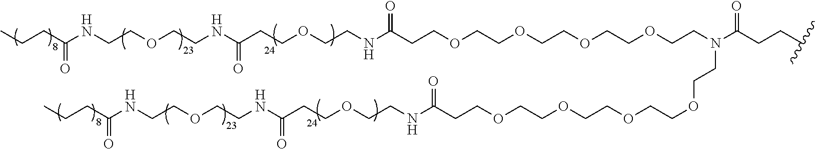

LP-29-a

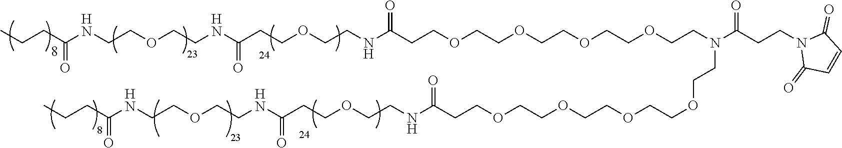

LP-29-p

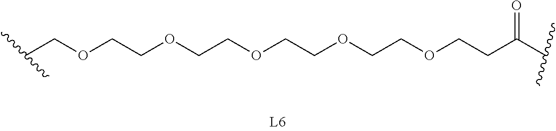 [SMILES: *COCCOCCOCCOCCOCCC(*)=O]

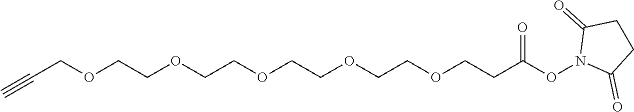 [SMILES: C#CCOCCOCCOCCOCCOCCC(=O)ON1C(=O)CCC1=O]

L6-p

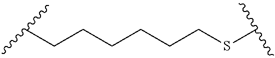 [SMILES: *CCCCCCS*]

(C6-S)

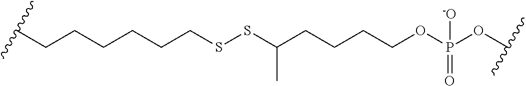

(C6-SS-MeC5)

Alternatively, other linking groups known in the art may be used. In many instances, linking groups can be commercially acquired or alternatively, are incorporated into commercially available nucleotide phosphoramidites.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents described herein can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors.

The above provided embodiments and items are now illustrated with the following, non-limiting examples.

### EXAMPLES

### Example 1. Synthesis of 5-((6-(bis(4-methoxyphenyl)(phenyl)methoxy)hexyl)disulfaneyl) hexyl(2-cyanoethyl) diisopropylphosphoramidite

Compound 8 (i.e., 5-((6-(bis(4-methoxyphenyl)(phenyl)methoxy)hexyl) disulfaneyl) hexyl(2-cyanoethyl) diisopropylphosphoramidite disclosed herein was synthesized in accordance with the following procedure:

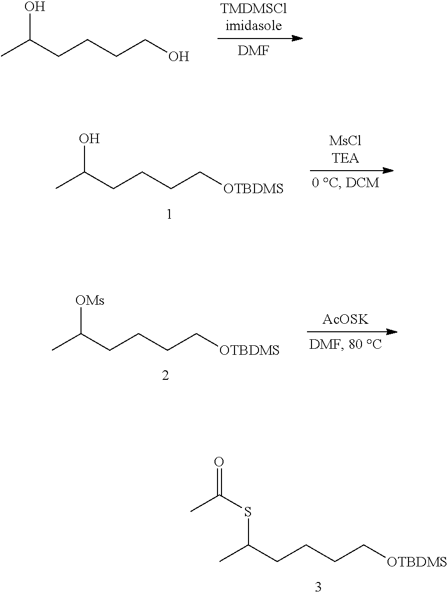

Compound 1:1,5-hexan-di-ol (1 ml, 8.47 mmol) was dissolved in anhydrous DMF (10 mL) and imidazole (576 mg, 8.47 mmol, 1 eq). TBDMSCl (1.28 g, 8.47 mmol., 1 eq) was added at 0° C. in 6 portions with 20 min intervals. Warmed to RT and stirred for 2 h and monitored by TLC (KMnO4 stain). Reaction was quenched with water and stirred for additional 10 min. 10 ml of brine was added and extracted with EtOAc (3×50 ml). Organic phases were washed with brine and dried over Na₂SO₄. Evaporated thoroughly (removed also traces of DMF). Loaded on 40 g SG column and eluted with HEX/EtOAc (Washed @ ~35% EtOAc). 1.22 g of compound 1 was isolated (60% yield).

Compound 2: Compound 1 (1.2 g, 5.16 mmol) was dissolved in DCM (50 mL) and cooled to 0° C. TEA (1.4 mL, 10.3 mmol, 2.0 eq) was added. A solution of MsCl in anhydrous DCM (2 ml) was then added. Reaction was carried out at 0° C. for 1 h and allowed to warm to RT. (15 min) TLC showed no SM. Reaction was quenched with water (1 mL) and stirred for 5 min. DCM and citric acid were added and organic phase was washed with citric acid (2×20 ml) and 30 ml of NaHCO₃. Dried with sodium sulfate, concentrated and loaded on SG column eluting with hex:EtOAc (washed @~25% EtOAc). 1.33 g of mesylate 2 was obtained (83% yield).

Compound 3: Compound 2 (800 mg, 2.58 mmol), potassium thioacetate (1.17 g, 10.3 mmol, 4 eq) and potassium iodide (80 mg, 0.2 eq) were suspended in DMF (17 ml) and heated at 80° C. No SM after 40 min. Reaction was cooled to RT and partitioned between sol. of sodium bicarbonate and TBME (90 ml). Water phase was extracted with another 90 ml of TBME and combined organic phases were washed with sodium bicarbonate. Organic phase was dried and concentrated. Loaded on 40 g column and eluted with hex:AcOEt (washed @15% EtOAc). 738 mg of brown liquid was collected (98% yield).

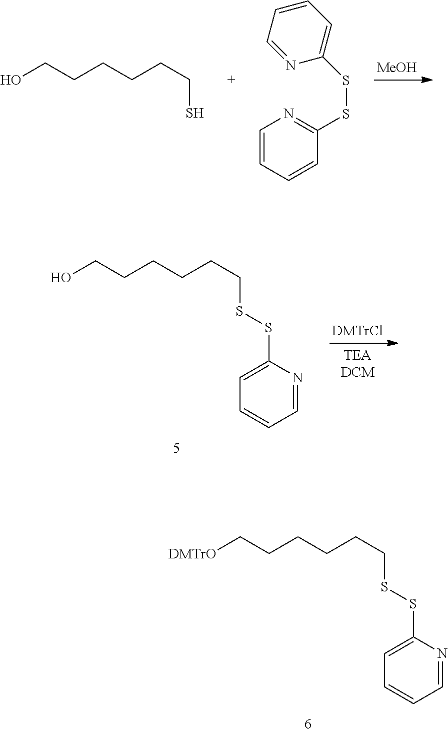

Compound 5: Dithiopyridyl (3.38 g, 15.35 mmol, 3 eq) was dissolved in MeOH (14 mL) and mixture was degassed. 1-mercaptohexan-6-ol (700 μL, 5.12 mmol, 1 eq) and mixture was stirred overnight under N₂. MeOH was evaporated and remaining oil was evaporated with DCM 2 times. Loaded on 80 g SG column. Isolated compound 5, as sticky oil (quantitative yield).

Compound 6: Disulfide 5 (600 mg, 2.5 mmol) was dissolved in dry DCM (21 ml) and TEA (0.9 mL, 6.6 mmol, 2.5 eq) was added. A solution of DMTrCl (910 mg, 2.7 mmol, 1.1 eq) in DCM (2 mL) was added dropwise and mixture was left overnight at RT. MeOH (1 mL) was added and mixture was stirred for 10 min. Concentrated, dried to remove MeOH and loaded on 120 g SG column (conditioned with 1% TEA, washed with 0.5% TEA/hexan:AcOEt). Washed @ ~30% AcOEt. 1.3 g (73% was isolated).

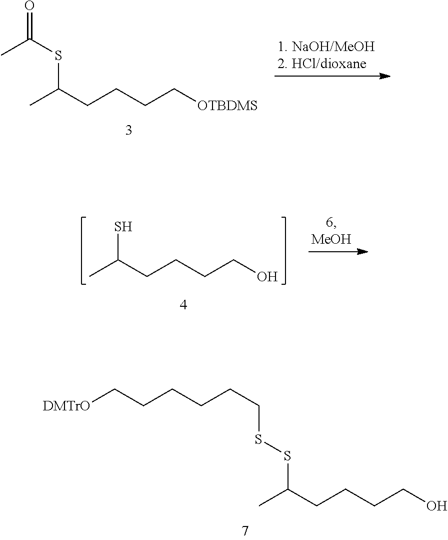

Compound 4: Thioacetate (3, 369 mg, 1.27 mmol) was dissolved in 9 ml of MeOH and degassed using N₂ bubbling/sonication cycles (3×). NaOH in MeOH (35 mg/ml, 3 mL) was added and the reaction was stirred for 2 h under nitrogen. NaOH was quenched with HCl/dioxane (0.6 ml) and stirred for 15 min. pH was adjusted to ~10 with TEA.

Compound 7: The suspension described above was then filtered directly to a compound 6 (1.04 g, 1.91 mmol, 1.5 eq) in MeOH/DCM (~4:2, 8 mL). Stirred overnight at RT. TLC showed 3 major spots (the middle one should be compound 7). Solvents were evaporated and resulting oil was evaporated with DCM few times to get rid of residual MeOH. Loaded on 80 g SG column (conditioned with 1% TEA/hexane, washed with 0.5% TEA/hexane). First fraction is an unreacted 6, followed by compound 7 (@~40% AcOEt) and finally yellowish thio-pyridine. 509 mg was isolated (71%).

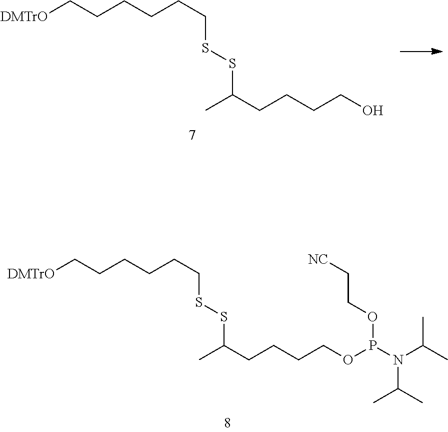

5-((6-(bis(4-methoxyphenyl)(phenyl)methoxy)hexyl)disulfaneyl) hexyl(2-cyanoethyl) diisopropylphosphoramidite (i.e., Compound 8, C6-SS-MeC5 phosphoramidite): Starting alcohol (509 mg, 0.89 mmol) was dissolved in 1 mL of dry DCM (Acros, sealed) and 4 mL of toluene and evaporated. Repeated 2 times. Dissolved in dry DCM (4 mL) with 4 A MS (3-4 beads). N,N-diisopropylammonium tetrazolide (169 mg, 0.98 mmol, 1.1 eq) was added and suspension was cooled ~5° C. Diamidite was dissolved in dry DCM (2 mL) and added into reaction dropwise over 15 mins. Allowed to RT. TLC showed some SM after 30 min but no SM after 50 min. Reaction was quenched with 2 mL of sat. NaHCO₃ (aq.) Water phase was extracted with 10 ml of DCM. Combined organic phases were washed with NaHCO₃ (2×5 mL), dried over sodium sulfate and loaded on SG column (20 g, conditioned with 1% TEA/hexane). Washed @~30% AcOEt yielding 440 mg (68%) of colorless oil.

### Example 2. Comparison of Phosphoramidite Stability in Acetonitrile

Oligonucleotide strand AM08891-SS was synthesized using freshly-prepared C6-SS-C6 amidite solution or a 24-hour aged C6-SS-C6 amidite solution and the results are shown in Table 2.2 below.

TABLE 2.1
Sense strands synthesized in Example 2.

| Sequence | Sense Sequence (5′→3′) | SEQ ID NO. |
|---|---|---|
| AM08891-SS | (NH2-C6)s(invAb)sggucaugaUfCfUfugcuguaacas(invAb)- | 1 |
| | (C6-SS-C6)dT | |
| AM08891-SS | (NH2-C6)s(invAb)sggucaugaUfCfUfugcuguaacas(invAb)- | 2 |
| | (C6-SS-MeC5)dT | |

TABLE 2.2
Yields for synthesis of AM08891-SS using fresh or aged C6-SS-C6

| | Fresh 0.2M | 24 hr Aged 0.2M |
|---|---|---|
| | C6-SS-C6 | C6-SS-C6 |
| Scale | 220 umol | 220 umol |
| Crude Yield | 62.3%, 1.12 g | 35.9%, 0.64 g |
| Crude Purity by Den AEX | 71.5% | 67.1% |

As can be seen in Table 2.2, aging C6-SS-C6 phosphoramidite for 24 hours in acetonitrile leads to a dramatic decrease in yield and purity for the oligonucleotide synthesis. Without wishing to be bound by any particular theory, it is believed that the C6-SS-C6 phosphoramidite is not stable in acetonitrile due to crosslinking in solution prior to coupling to the dT residue on the solid support. These cross-linking products (i.e., the disulfide bisphosphoramidite and the disulfide bisDMT shown in scheme 2.1 may hinder oligonucleotide elongation and reduce the overall yield of the oligonucleotide synthesis.

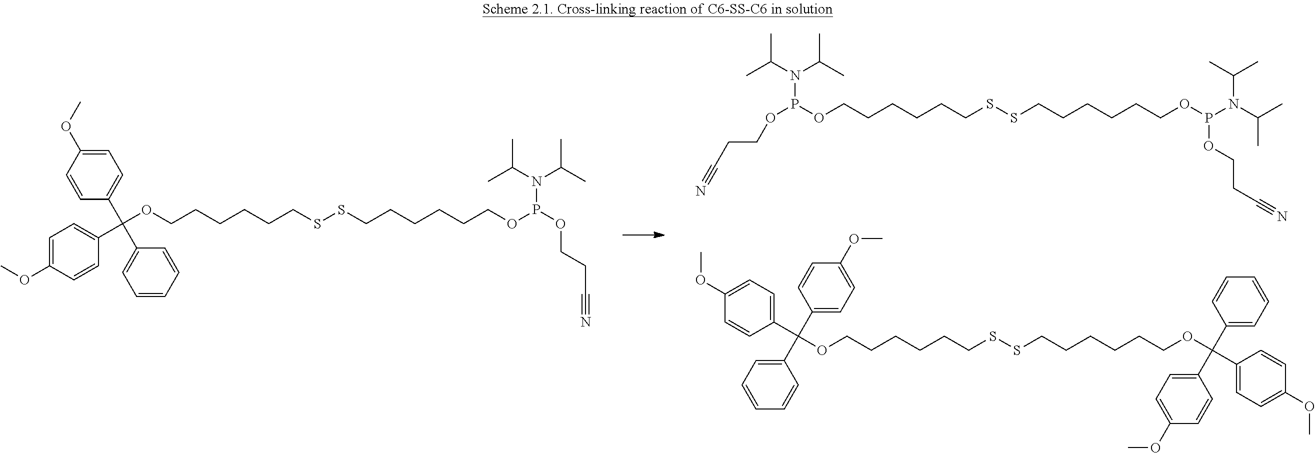 [SMILES: COc1ccc(C(OCCCCCCSSCCCCCCOP(OCCC#N)N(C(C)C)C(C)C)(c2ccccc2)c2ccc(OC)cc2)cc1 ; CC(C)N(C(C)C)P(OCCC#N)OCCCCCCSSCCCCCCOP(OCCC#N)N(C(C)C)C(C)C ; COc1ccc(C(OCCCCCCSSCCCCCCOC(c2ccccc2)(c2ccc(OC)cc2)c2ccc(OC)cc2)(c2ccccc2)c2ccc(OC)cc2)cc1]

Oligonucleotide strand AM08891-SS was synthesized using freshly-prepared amidite solution of Compound 8 or a 24-hour aged amidite solution of Compound 8 and the results are shown in Table 2.3 below.

TABLE 2.3
Yields for synthesis of AM08891-SS
using fresh or aged Compound 8

| | Fresh 0.2M | 24 hr aged 0.2M |
|---|---|---|
| | Compound 8 | Compound 8 |
| Scale | 220 μmol | 220 μmol |
| Crude Yield | 65.2%, 1.17 g | 61.5%, 1.10 g |
| Crude Purity by Den AEX | 73.6% | 72.7% |

As can be seen in Table 2.3, the yield and purity were similar between the fresh and aged batches of Compound 8, and the 24 hour aged solution performed much better when Compound 8 is used compared to the C6-SS-C6 phosphoramidite, giving a ~4% difference in yield compared to a ~26% drop in yield, respectively.

### Example 3. Comparison of Disulfide Stability During Cleavage/Deprotection Step

Oligonucleotide strand AM16135-SS was synthesized using freshly-prepared (C6-SS-C6), (C6-SS-MeC5), or a C12 spacer as the phosphoramidite, and the results are shown in Table 3.2 below. As can be seen in Table 3.2, phosphoramidites (C6-SS-MeC5) and C12 gave higher yields compared to when (C6-SS-C6) is used. In addition, no dimer formation was detected during the cleavage/deprotection step of the oligonucleotide synthesis (See scheme 3.1 showing dimer formation when (C6-SS-C6) is used).

TABLE 3.1
Sense strands synthesized in Example 3.

| Sequence | Sense Sequence (5′→3′) | SEQ ID NO. |
|---|---|---|
| AM16135-SS | (NH2-C6)scscugugcaAfCfCfagaacaaauas(invAb)(C6-SS- | 3 |
| | C6)dT | |
| AM16135-SS | (NH2-C6)scscugugcaAfCfCfagaacaaauas(invAb)(C6-SS- | 4 |
| | MeC5)dT | |
| AM16135-SS | (NH2-C6)scscugugcaAfCfCfagaacaaauas(invAb)(C12)dT | 5 |

TABLE 3.2
Yields for synthesis of AM16135-SS using
freshly prepared phosphoramidite

| | Yield | Yield | | | |
|---|---|---|---|---|---|
| Phosphoramidite | (g) | (g/mmol) | Yield % | Purity % | Dimer % |
| (C6-SS-C6) | 1.81 | 5.18 | 65 | 80 | 1.88 |
| (C6-SS-MeC5) | 1.94 | 5.54 | 69 | 81 | ND |
| C12 | 1.97 | 5.63 | 71 | 81 | ND |

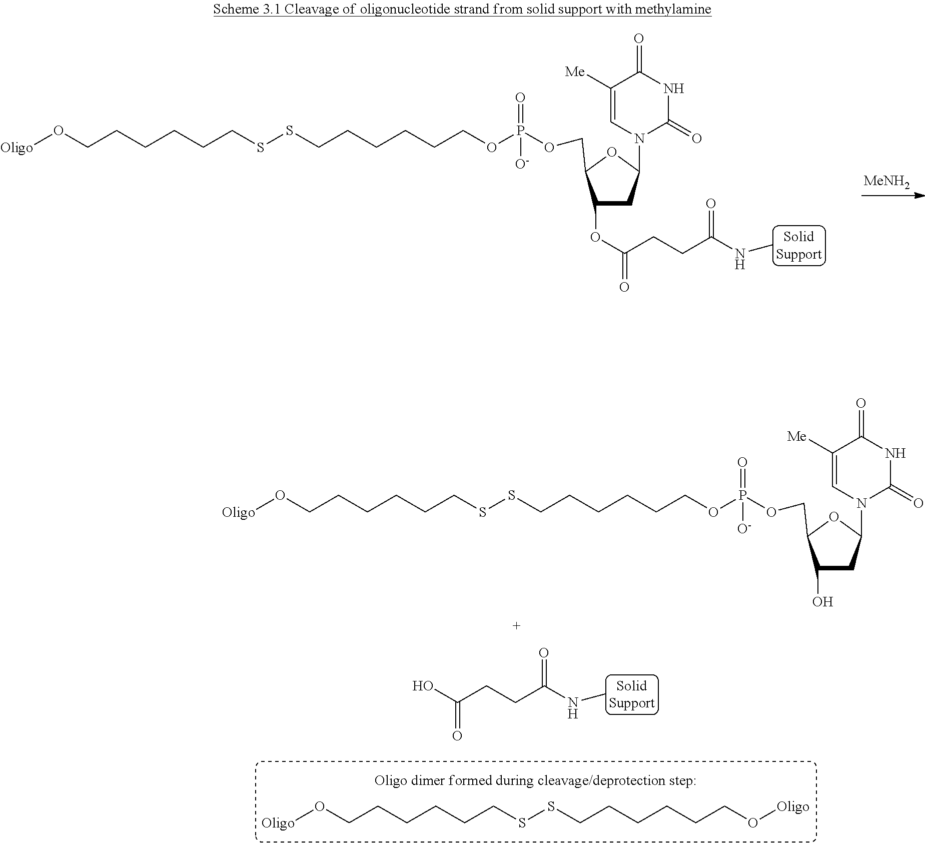

Synthesis of AM16135-SS was also carried out using an aged solution (aged 8 days in toluene) of either the (C6-SS-C6) or the (C6-SS-MeC5) phosphoramidites and these results compared to the respective fresh solutions (see tables 3.3 and 3.4). There is an increase in yield when the strand is synthesized using the (C6-SS-MeC5) phosphoramidite compared to the C6-SS-C6 phosphoramidite, and a decrease in purity between the fresh and aged toluene solution for the (C6-SS-C6) phosphoramidite, as well as an increased amount of oligonucleotide dimer formation during the cleavage/deprotection step.

TABLE 3.3
Yields for synthesis of AM16135-SS using
C6-SS-C6, fresh or aged in toluene

| | Yield (g) | Yield (g/mmol) | Yield % | Purity % | Dimer % |
|---|---|---|---|---|---|
| Day 1 | 1.81 | 5.18 | 65 | 80 | 1.88 |
| Day 8 | 1.83 | 5.22 | 65 | 75 | 2.25 |

TABLE 3.4
Yields for synthesis of AM16135-SS using
(C6-SS-MeC5), fresh or aged in toluene

| | Yield (g) | Yield (g/mmol) | Yield % | Purity % | Dimer % |
|---|---|---|---|---|---|
| Day 1 | 1.94 | 5.54 | 69 | 81 | ND |
| Day 8 | 1.96 | 5.59 | 70 | 78 | ND |

### Example 4. Comparison of Disulfide Stability Using Different Cleavage Conditions

The synthesis of oligonucleotide strand AM09965-SS was carried out using a fresh or aged solution of C6-SS-C6 phosphoramidite prepared in toluene or a fresh or aged solution of (C6-SS-MeC5) phosphoramidite prepared in acetonitrile. After the synthesis of AM09965-SS on the column was complete using the different phosphoramidite solutions, different cleavage conditions were used to cleave the oligonucleotide off the solid support (either 50 mL of 40% MeNH₂/mmol or 500 mL of 20% MeNH₂/mmol). The results of these various conditions are shown in Table 4.2 below.

TABLE 4.1
Sense strands synthesized in Example 4.

| Sequence | Sense Sequence (5′→3′) | SEQ ID NO. |
|---|---|---|
| AM09965-SS | (NH2-C6)s(invAb)scaggauucAfGfAfucugguuucas(invAb) | 6 |
| | (C6-SS-C6)dT | |
| AM09965-SS | (NH2-C6)s(invAb)scaggauucAfGfAfucugguuucas(invAb) | 7 |
| | (C6-SS-MeC5)dT | |

TABLE 4.1
Comparison of various cleavage/deprotection conditions

| | | Crude - | Crude - | Crude - |
|---|---|---|---|---|
| | Cleavage | FLP Yield | FLP | FLP Yield |
| Disulfide/Condition | conditions | (%) | Purity | (g)* |
| (C6-SS-C6)/Fresh | 40% MeNH₂ | 32 | 50 | 0.660 |
| (C6-SS-MeC5)/Fresh | 40% MeNH₂ | 62 | 88 | 1.271 |
| (C6-SS-C6)/Aged | 40% MeNH₂ | 34 | 53 | 0.693 |
| (C6-SS-MeC5)/Aged | 40% MeNH₂ | 57 | 87 | 1.169 |
| (C6-SS-C6)/Fresh | 20% MeNH₂ | 58 | 90 | 1.189 |
| (C6-SS-MeC5)/Fresh | 20% MeNH₂ | 56 | 89 | 1.149 |
| (C6-SS-C6)/Aged | 20% MeNH₂ | 55 | 87 | 1.130 |
| (C6-SS-MeC5)/Aged | 20% MeNH₂ | 61 | 91 | 1.243 |

As can be seen in Table 4.2, using 50 mL of 40% MeNH₂/mmol to cleave the oligonucleotide off the column resulted in much lower yields when the (C6-SS-C6) phosphoramidite was used (giving 32% yield for fresh phosphoramidite or 34% yield when the (C6-SS-C6) aged in toluene solution was used) compared to when the (C6-SS-MeC5) phosphoramidite is used (62% for fresh (C6-SS-MeC5) or 57% for (C6-SS-MeC5) aged in acetonitrile). There is also an increase in purity for the (C6-SS-MeC5) disulfide as well 88/87% compared to 50/53% purity.

Using more dilute cleavage conditions (i.e., 500 mL of 20% MeNH₂/mmol) gives more similar results between the two disulfides; however, this has limited commercial viability because roughly 5× more methylamine is used leading to much more waste and expense.

### Example 5. Conjugation of Lipid PK/PD Modulator Precursors

Either prior to or after annealing and prior to or after conjugation of one or more targeting ligands, one or more lipid PK/PD modulator precursors can be linked to an oligonucleotide-based agent. The following describes the general conjugation process used to link lipid PK/PD modulator precursors to the RNAi agent constructs set forth in the Examples depicted herein.

### A. Conjugation of Activated Ester PK/PD Modulators

The following procedure was used to conjugate PK/PD modulators having an activated ester moiety such as TFP (tetrafluorophenoxy) or PNP (para-nitrophenol) to an RNAi agent with an amine-functionalized sense strand, such as C6-NH2, NH2-C6, or (NH2-C6). An annealed RNAi Agent dried by lyophilization was dissolved in DMSO and 10% water (v/v %) at 25 mg/mL. Then 50-100 equivalents of TEA and 3 equivalents of activated ester PK/PD modulator were added to the solution. The solution was allowed to react for 1-2 hours, while monitored by RP-HPLC-MS (mobile phase A 100 mM HFIP, 14 mM TEA; mobile phase B: acetonitrile on an Waters™ XBridge C18 column, Waters Corp.)

The product was then precipitated by adding 12 mL acetonitrile and 0.4 mL PBS and centrifuging the solid to a pellet. The pellet was then re-dissolved in 0.4 mL of 1×PBS and 12 mL of acetonitrile. The resulting pellet was dried on high vacuum for one hour.

### B. Conjugation of a Maleimide-Containing Lipid PK/PD Modulator Precursor

The following describes the general process used to link a maleimide-containing lipid PK/PD modulator precursor to a disulfide functionalized (e.g., (C6-SS-C6) or (C6-SS-MeC5) sense strand of an RNAi agent by undertaking a dithiothreitol reduction of disulfide followed by a thiol-Michael Addition of the respective maleimide-containing lipid PK/PD modulator precursor. In a vial, the functionalized sense strand was dissolved at 50 mg/mL in sterilized water. Then 20 equivalents of each of 0.1M Hepes pH 8.5 buffer and dithiothreitol were added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the maleimide-containing lipid PK/PD modulator precursor was added at 1.5 equivalents. The mixture was allowed to react for 30 minutes. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

### C. Conjugation of a Sulfone-Containing Lipid PK/PD Modulator Precursor

The following describes the general process used to link a sulfone-containing lipid PK/PD modulator precursor to a disulfide functionalized (e.g., (C6-SS-C6) or (C6-SS-MeC5) sense strand of an RNAi agent by undertaking a dithiothreitol reduction of disulfide followed by a substitution reaction of the respective sulfone-containing lipid PK/PD modulator precursor.

In a vial, functionalized sense strand was dissolved at 50 mg/mL in sterilized water. Then 20 equivalents of each of 0.1M Hepes pH 8.5 buffer and dithiothreitol are added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the sulfone-containing lipid PK/PD modulator precursor was added at 1.5 equivalents. The vial was purged with N₂, and heated to 40° C. while stirring. The mixture was allowed to react for one hour. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

### D. Conjugation of an Azide-Containing Lipid PK/PD Modulator Precursor

One molar equivalent of TG-TBTA resin loaded with Cu (I) was weighed into a glass vial. The vial was purged with N₂ for 15 minutes. Then, functionalized sense strand was dissolved in a separate vial in sterilized water at a concentration of 100 mg/mL. Then two equivalents of the azide-containing lipid PK/PD modulator precursor (50 mg/mL in DMF) is added to the vial. Then TEA, DMF and water are added until the final reaction conditions are 33 mM TEA, 60% DMF, and 20 mg/mL of the conjugated product. The solution was then transferred to the vial with resin via a syringe. The N₂ purge was removed and the vial was sealed and moved to a stir plate at 40° C. The mixture was allowed to react for 16 hours. The resin was filtered off using a 0.45 μm filter.

The product was purified using AEX purification (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile solid phase TSKgel-30; 1.5 cm×10 cm.) The acetonitrile was removed using a rotary evaporator, and desalted with a 3K spin column using 2×10 mL exchanges with sterilized water. The solid product was dried using lyophilization and stored for later use.

### Example 6. Synthesis of RNAi Agents and Compositions

The following describes the general procedures for the syntheses of the oligonucleotide-based agents, such as RNAi agents and antisense oligonucleotides, and conjugates thereof as described herein.

Synthesis of Oligonucleotide-based Agents. Oligonucleotide-based agents can be synthesized using methods generally known in the art. For the synthesis of the RNAi agents illustrated in the Examples set forth herein, the sense and antisense strands of the RNAi agents were synthesized according to solid phase phosphoramidite technology used in oligonucleotide synthesis. Depending on the scale, a MerMade96E® (Bioautomation), a MerMade12® (Bioautomation), or an Oligopilot 100 (GE Healthcare) was used. Syntheses were performed on a solid support made of controlled pore glass (CPG, 500 Å or 600 Å, obtained from Prime Synthesis, Aston, PA, USA) or polystyrene (obtained from Kinovate, Oceanside, CA, USA). All RNA and 2′-modified RNA phosphoramidites were purchased from Thermo Fisher Scientific (Milwaukee, WI, USA), ChemGenes (Wilmington, MA, USA), or Hongene Biotech (Morrisville, NC, USA). Specifically, the following 2′-O-methyl phosphoramidites that were used include the following: (5′-O-dimethoxytrityl-N⁶-(benzoyl)-2′-O-methyl-adenosine-3′-O-(2-cyanoethyl-N,N-diisopropylamino)phosphoramidite, 5′-O-dimethoxy-trityl-N⁴-(acetyl)-2′-O-methyl-cytidine-3′-O-(2-cyanoethyl-N,N-diisopropyl-amino)phosphoramidite, (5′-O-dimethoxytrityl-N2-(isobutyryl)-2′-O-methyl-guanosine-3′-O-(2-cyanoethyl-N,N-diisopropylamino)phosphoramidite, and 5′-O-dimethoxytrityl-2′-O-methyl-uridine-3′-O-(2-cyanoethyl-N,N-diisopropylamino)phosphoramidite. The 2′-deoxy-2′-fluoro-phosphoramidites and 2′-O-propargyl phosphoramidites carried the same protecting groups as the 2′-O-methyl phosphoramidites. 5′-dimethoxytrityl-2′-O-methyl-inosine-3′-O-(2-cyanoethyl-N,N-diisopropylamino)phosphoramidites were purchased from Glen Research (Virginia). The inverted abasic (3′-O-dimethoxytrityl-2′-deoxyribose-5′-O-(2-cyanoethyl-N,N-diisopropylamino)phosphoramidites were purchased from ChemGenes. The following UNA phosphoramidites that were used included the following: 5′-(4,4′-Dimethoxytrityl)-N⁶-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-di-iso-propyl)]-phosphoramidite. In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile or a 200 mM solution of xanthane hydride (TCI America, Portland, OR, USA) in pyridine was employed. Antisense oligonucleotides may be manufactured using the same general methods as those used in connection with the RNAi agent conjugates described in the Examples herein.

TFA aminolink phosphoramidites were also commercially purchased (ThermoFisher). Linker L6 was purchased as propargyl-PEG5-NHS from BroadPharm (catalog #BP-20907) and coupled to the NH2-C6 group from an aminolink phosphoramidite to form-L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker-Alk-cyHex-. In each case, phosphorothioate linkages were introduced as specified using the conditions set forth herein.

For some RNAi agents disclosed herein, a linker, such as a C6-SS-C6 or a C6-SS-MeC5 group, was introduced at the 3′ terminal end of the sense strand. For some sense strands, a dT resin (e.g., NittoPhase™ HL) was used and the respectively linker was then added to the dT residue of the solid support resin.

Cleavage and deprotection of support bound oligomer. After finalization of the solid phase synthesis, the dried solid support was treated with 50 mL of 40 weight (wt.) % methylamine in water per mmol of solid support for 100 min at room temperature. The solution was cooled in an iced bath, diluted with water, then filtered.

Purification. Crude oligomers may be purified by anionic exchange HPLC using a TSKgel® SuperQ-5 PW 13 μm column (available from Tosoh Biosciences) and Shimadzu LC-8 system. Appropriate fractions may be pooled and then run on size exclusion HPLC using a GE Healthcare XK 16/40 column packed with Sephadex® G25 fine (available from Sigma Aldrich) with a running buffer of 100 mM ammonium bicarbonate, pH 6.7 and 20% Acetonitrile or filtered water. Alternatively, pooled fractions may be desalted and exchanged into an appropriate buffer or solvent system via tangential flow filtration.

Annealing. For the RNAi agents disclosed in the Examples herein, complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at ~15 to −25° C. Duplex concentration was determined by measuring the solution absorbance on a UV-Vis spectrometer in 1×PBS. The solution absorbance at 260 nm was then multiplied by a conversion factor and the dilution factor to determine the duplex concentration. The conversion factor used was either 0.037 mg/(mL·cm) or was calculated from an experimentally determined extinction coefficient.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Embodiment 1. A functionalized solid support for oligonucleotide synthesis comprising the structure:

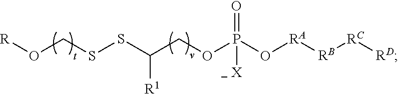

wherein R is hydrogen, a modified or unmodified nucleotide, a modified or unmodified oligonucleotide, RNAi agent, or an oxygen protecting group;
R¹ is substituted or unsubstituted C₁-C₆ alkyl;
X is O or S;
R^{A} is a modified or unmodified nucleotide;
R^{B} is a linker;
R^{C} is a spacer;
R^{D} is a solid support material
t is an integer between 1 and 20, inclusive; and
v is an integer between 1 and 20, inclusive.

Embodiment 2. The compound of embodiment 1, wherein t is 3, 4, 5, 6, 7, or 8.

Embodiment 3. The compound of embodiment 1 or 2, wherein v is 2, 3, 4, 5, or 6.

Embodiment 4. A functionalized solid support for oligonucleotide synthesis comprising the structure:

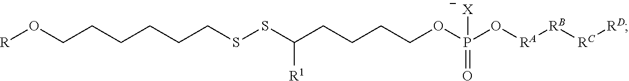

wherein R is hydrogen, a modified or unmodified nucleotide, a modified or unmodified oligonucleotide, RNAi agent, or an oxygen protecting group;
R¹ is substituted or unsubstituted C₁-C₆ alkyl;
X is O or S;
R^{A} is a modified or unmodified nucleotide;
R^{B} is a linker;
R^{C} is a spacer; and
R^{D} is a solid support material.

Embodiment 5. The solid support of any one of embodiments 1-4, wherein R^{A} is modified or unmodified adenosine nucleotide, modified or unmodified cytidine nucleotide, modified or unmodified guanosine nucleotide, modified or unmodified uridine nucleotide, modified or unmodified inosine nucleotide, or modified or unmodified deoxythymidine nucleotide.

Embodiment 6. The solid support of any one of embodiments 1-5, wherein R^{A} is modified or unmodified deoxythymidine nucleotide.

Embodiment 7. The solid support of any one of embodiments 1-6 comprising the structure:

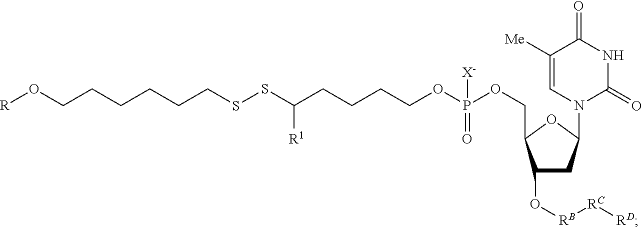

wherein R is hydrogen, a modified or unmodified nucleotide, or an oxygen protecting group;
R¹ is substituted or unsubstituted C₁-C₆ alkyl;
X is O or S;
R^{B} is a linker;
R^{C} is a spacer; and
R^{D} is a solid support material.

Embodiment 8. The solid support of any one of embodiments 1-7, wherein R^{B} is of the formula:

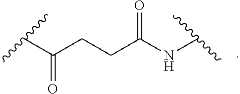 [SMILES: *NC(=O)CCC(*)=O]

Embodiment 9. The solid support of any one of embodiments 1-8, comprising the structure:

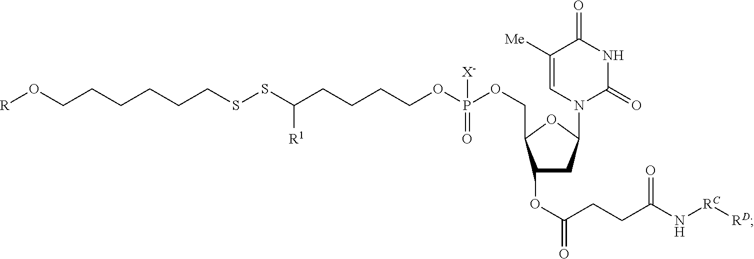

wherein R is hydrogen, a modified or unmodified nucleotide, or an oxygen protecting group;
R¹ is substituted or unsubstituted C₁-C₆ alkyl;
X is O or S;
R^{C} is a spacer; and
R^{D} is a solid support material.

Embodiment 10. The solid support of any one of embodiments 1-9, wherein R^{C} is of the formula:

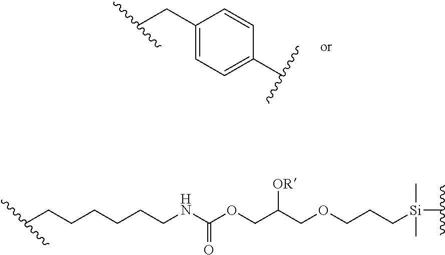

wherein R′ is C₁-C₆ alkyl.

Embodiment 11. The solid support of any one of embodiments 1-10 comprising the structure:

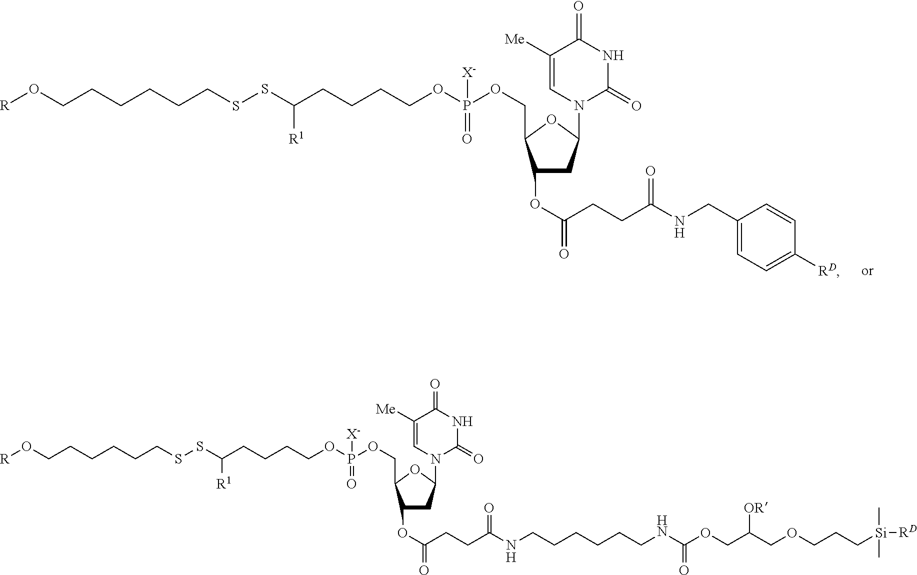

wherein R is hydrogen, a modified or unmodified nucleotide, a modified or unmodified oligonucleotide, RNAi agent, or an oxygen protecting group;
X is O or S;
R¹ is substituted or unsubstituted C₁-C₆ alkyl;
R′ is C₁-C₆ alkyl; and
R^{D} is a solid support material.

Embodiment 12. The solid support of any one of embodiments 1-11, wherein the solid support material comprises polystyrene or controlled pore glass (CPG).

Embodiment 13. The solid support of any one of embodiments 1-12, wherein R is an oxygen protecting group.

Embodiment 14. The solid support of any one of embodiments 1-13, wherein R is a dimethoxytrityl (DMT) protecting group.

Embodiment 15. The solid support of any one of embodiments 1-12, wherein R is a modified or unmodified nucleotide.

Embodiment 16. The solid support of embodiment 15, wherein the oligonucleotide comprises one or more modified nucleotides.

Embodiment 17. The solid support of embodiment 16, wherein the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 18. The solid support of any one of embodiments 15-17, wherein the oligonucleotide comprises one or more modified internucleoside linkages.

Embodiment 19. The solid support of any one of embodiments 15-18, wherein the oligonucleotide comprises a sequence that is between 18 and 30 nucleotides in length.

Embodiment 20. The solid support of embodiment 19, wherein the oligonucleotide comprises a sequence that is between 18 and 24 nucleotides in length.

Embodiment 21. The solid support of embodiment 19 or 20, wherein the oligonucleotide comprises a sequence that is 21 nucleotides in length.

Embodiment 22. The solid support of any one of embodiments 1-21, wherein R¹ is substituted or unsubstituted methyl.

Embodiment 23. The solid support of any one of embodiments 1-22, wherein R¹ is unsubstituted methyl.

Embodiment 24. The solid support of any one of embodiments 1-23, wherein X is O.

Embodiment 25. The solid support of any one of embodiments 1-24, wherein X is S.

Embodiment 26. A compound of the Formula (III′):

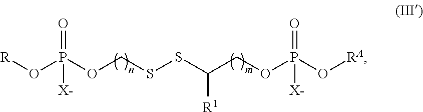

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S;
R¹ is substituted or unsubstituted C₁-C₆ alkyl;
n is an integer between 0 and 20, inclusive;
and m is an integer between 0 and 20, inclusive.

Embodiment 27. The compound of embodiment 26, wherein n is 6.

Embodiment 28. The compound of embodiment 26 or 27, wherein m is 4.

Embodiment 29. A compound of the Formula (III):

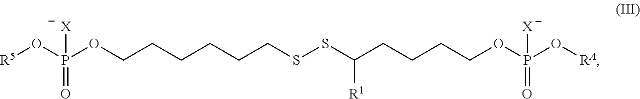

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S; and
R¹ is substituted or unsubstituted C₁-C₆ alkyl.

Embodiment 30. A compound of the Formula (IV′):

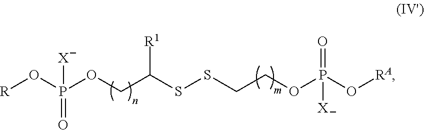

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S;
R¹ is substituted or unsubstituted C₁-C₆ alkyl;
n is an integer between 0 and 20, inclusive;
and m is an integer between 0 and 20, inclusive.

Embodiment 31. The compound of embodiment 30, wherein n is 4.

Embodiment 32. The compound of embodiment 30 or 31, wherein m is 5.

Embodiment 33. A compound of the Formula (IV):

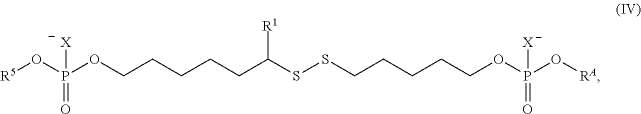

or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S; and
R¹ is substituted or unsubstituted C₁-C₆ alkyl.

Embodiment 34. The compound of any one of embodiments 26-33, wherein at least one instance of X is S.

Embodiment 35. The compound of any one of embodiments 26-33, wherein at least one instance of X is O.

Embodiment 36. The compound of any one of embodiments 26-35, wherein R^{A} is a modified or unmodified nucleotide.

Embodiment 37. The compound of any one of embodiments 26-36, wherein R^{A} is modified or unmodified adenosine, modified or unmodified cytidine, modified or unmodified guanosine, modified or unmodified uridine, modified or unmodified inosine, or modified or unmodified thymidine.

Embodiment 38. The compound of any one of embodiments 26-37, wherein R^{A} is modified or unmodified thymidine.

Embodiment 39. The compound of any one of embodiments 26-38, wherein R¹ is substituted or unsubstituted methyl.

Embodiment 40. The compound of any one of embodiments 26-39, wherein R¹ is unsubstituted methyl.

Embodiment 41. The compound of any one of embodiments 26-29, or 34-40, of the formula:

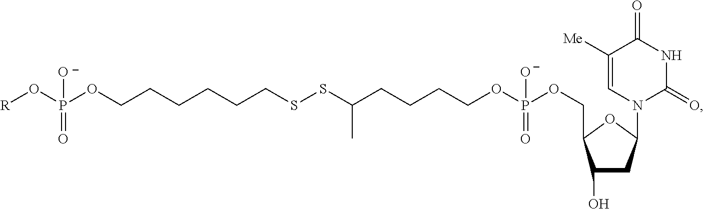

or a salt thereof.

Embodiment 42. The compound of any one of embodiments 30-40, of the formula:

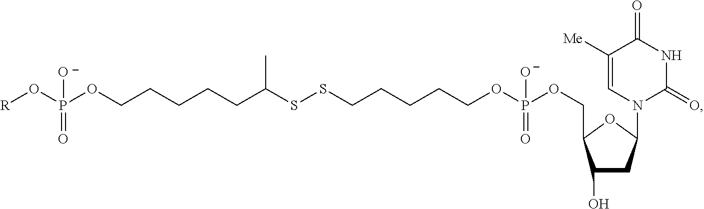

or a salt thereof.

Embodiment 43. The compound of any one of embodiments 26-42, wherein R is a modified or unmodified oligonucleotide.

Embodiment 44. The compound of embodiment 43, wherein the oligonucleotide comprises one or more modified nucleotides.

Embodiment 45. The compound of embodiment 44, wherein the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 46. The compound of any one of embodiments 43-45, wherein the oligonucleotide comprises one or more modified internucleoside linkages.

Embodiment 47. The compound of any one of embodiments 43-46, wherein the oligonucleotide comprises a sequence that is between 18 and 30 nucleotides in length.

Embodiment 48. The compound of embodiment 47, wherein the oligonucleotide comprises a sequence that is between 18 and 24 nucleotides in length.

Embodiment 49. The compound of embodiment 47 or 48, wherein the oligonucleotide comprises a sequence that is 21 nucleotides in length.

Embodiment 50. A compound of the Formula (I′):

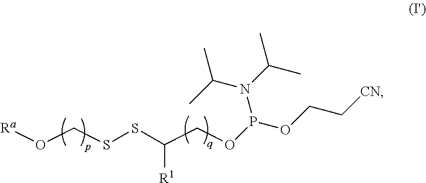

or a salt thereof, wherein:
R^{a} is hydrogen, a modified or unmodified nucleotide, an RNAi agent, or an oxygen protecting group;
R¹ is substituted or unsubstituted C₁-C₆ alkyl;
p is an integer between 0 and 20, inclusive; and
q is an integer between 0 and 20, inclusive.

Embodiment 51. The compound of embodiment 50, wherein p is 6.

Embodiment 52. The compound of embodiment 50 or 51, wherein q is 4.

Embodiment 53. A compound of the Formula (I):

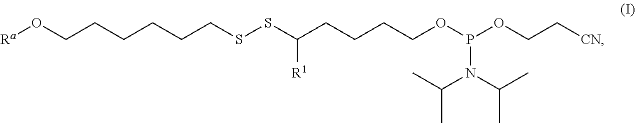

or a salt thereof, wherein:
R^{a} is hydrogen, a modified or unmodified nucleotide, an RNAi agent, or an oxygen protecting group; and R¹ is substituted or unsubstituted C₁-C₆ alkyl.

Embodiment 54. A compound of the Formula (V′):

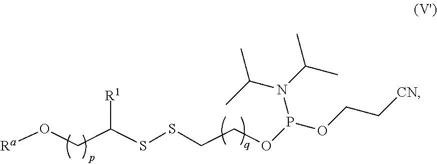

or a salt thereof, wherein:
R^{a} is hydrogen, a modified or unmodified nucleotide, an RNAi agent, or an oxygen protecting group;
R¹ is substituted or unsubstituted C₁-C₆ alkyl;
p is an integer between 0 and 20, inclusive; and
q is an integer between 0 and 20, inclusive.

Embodiment 55. The compound of embodiment 54, wherein p is 4.

Embodiment 56. The compound of embodiment 54 or 55, wherein q is 5.

Embodiment 57. A compound of the Formula (V):

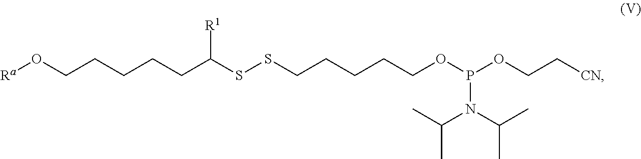

or a salt thereof, wherein:
R^{a} is hydrogen, a modified or unmodified nucleotide, an RNAi agent, or an oxygen protecting group; and R¹ is substituted or unsubstituted C₁-C₆ alkyl.

Embodiment 58. The compound of any one of embodiments 50-57, wherein R^{a} is an oxygen protecting group.

Embodiment 59. The compound of any one of embodiments 50-58, wherein R^{a} is a dimethoxytrityl (DMT) protecting group.

Embodiment 60. The compound of any one of embodiments 50-53, or 58-59, of the formula:

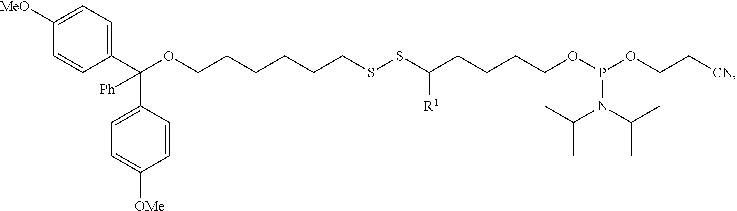

or a salt thereof wherein R¹ is substituted or unsubstituted C₁-C₆ alkyl.

Embodiment 61. The compound of any one of embodiments 54-59, of the formula:

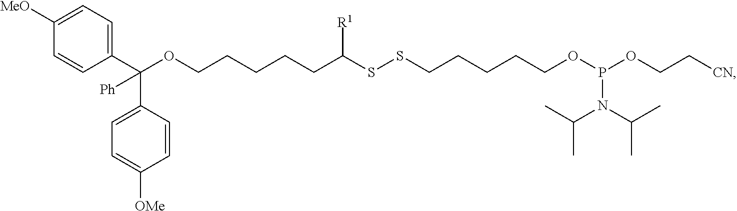

or a salt thereof wherein R¹ is substituted or unsubstituted C₁-C₆ alkyl.

Embodiment 62. The compound of any one of embodiments 50-61, wherein R¹ is substituted or unsubstituted methyl.

Embodiment 63. The compound of any one of embodiments 50-62, wherein R¹ is unsubstituted methyl.

Embodiment 64. The compound of any one of embodiments 50-53, 58-60, or 62-63, of the formula:

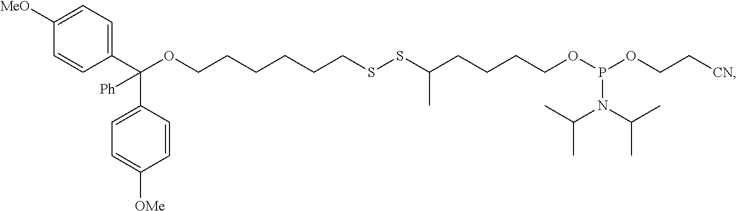 [SMILES: COc1ccc(C(OCCCCCCSSC(C)CCCCOP(OCCC#N)N(C(C)C)C(C)C)(c2ccccc2)c2ccc(OC)cc2)cc1]

or a salt thereof.

Embodiment 65. The compound of any one of embodiments 54-59, or 61-63, of the formula:

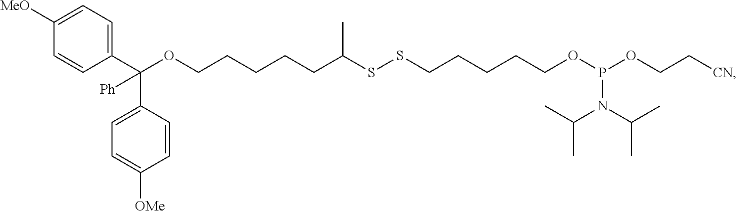 [SMILES: COc1ccc(C(OCCCCCC(C)SSCCCCCOP(OCCC#N)N(C(C)C)C(C)C)(c2ccccc2)c2ccc(OC)cc2)cc1]

or a salt thereof.

Embodiment 66. A compound of Formula (II):

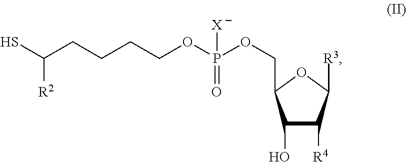

or a salt thereof, wherein:
R² is substituted or unsubstituted C₁-C₆ alkyl;
X is O or S;
R³ is a nucleobase; and
R⁴ is hydrogen, methoxy, or a halogen.

Embodiment 67. The compound of embodiment 66, wherein R⁴ is hydrogen.

Embodiment 68. The compound of embodiment 66 or 67, wherein R³ is cytosine, guanine, adenine, thymine, or uracil.

Embodiment 69. The compound of any one of embodiments 66-68, wherein R³ is thymine.

Embodiment 70. The compound of any one of embodiments 66-69, wherein R² is substituted or unsubstituted methyl.

Embodiment 71. The compound of any one of embodiments 66-70, wherein R² is unsubstituted methyl.

Embodiment 72. The compound of any one of embodiments 66-71, of the formula:

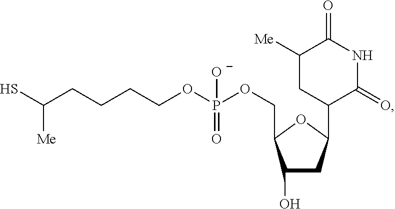

or a salt thereof.

Embodiment 73. A method comprising the step of reacting a support bound oligonucleotide comprising the structure:

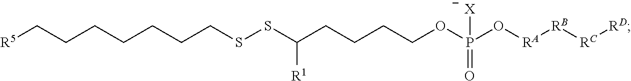

wherein R⁵ is a modified or unmodified oligonucleotide, or an RNAi agent;
R¹ is substituted or unsubstituted C₁-C₆ alkyl;
R^{A} is a modified or unmodified nucleotide;
R^{B} is a linker;
R^{C} is a spacer; and
R^{D} is a solid support material;
with an organic base to form an oligonucleotide of the formula:

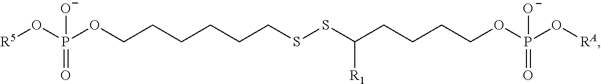

or a salt thereof.

Embodiment 74. The method of embodiment 73, wherein R^{B} is of the formula:

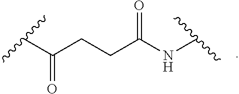 [SMILES: *NC(=O)CCC(*)=O]

Embodiment 75. The method of embodiment 73 or 74, wherein R^{C} is of the formula:

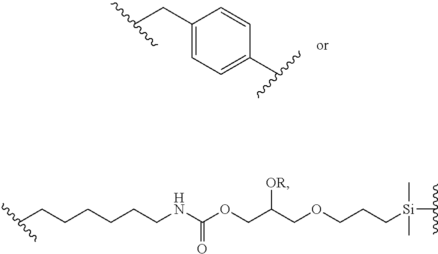

wherein R′ is C₁-C₆ alkyl.

Embodiment 76. The method of any one of embodiments 73-75, wherein the solid support material comprises polystyrene or controlled pore glass (CPG).

Embodiment 77. The method of any one of embodiments 73-76, wherein the organic base is an amine base.

Embodiment 78. The method of any one of embodiments 73-77, wherein the organic base is an alkylamine.

Embodiment 79. The method of any one of embodiments 73-78, wherein the organic base is methylamine.

Embodiment 80. A method comprising the steps of reacting a compound of the formula:

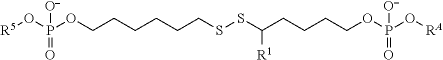

wherein R⁵ is a modified or unmodified oligonucleotide,
R¹ is substituted or unsubstituted C₁-C₆ alkyl, and
R^{A} is a modified or unmodified nucleotide, or an RNAi agent;
with a reducing agent to form a compound of the formula:

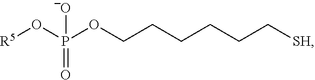

or a salt thereof.

Embodiment 81. The method of embodiment 80, further comprising the step of reacting the compound of the formula:

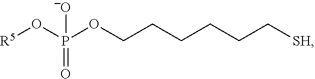

or a salt thereof wherein R⁵ is a modified or unmodified oligonucleotide, or an RNAi agent, with a compound of the formula:

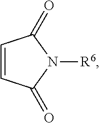

wherein R⁶ is a PK/PD modulator, to form a compound of the formula:

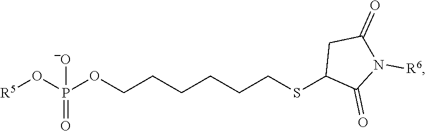

or a salt thereof.

Embodiment 82. The method of embodiment 80, further comprising the step of reacting the compound of the formula:

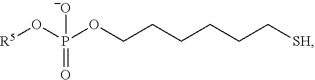

or a salt thereof, wherein R⁵ is a modified or unmodified oligonucleotide with a compound of the formula:

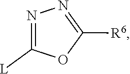

wherein: R⁶ is a PK/PD modulator, and L is a leaving group;
to form a compound of the formula:

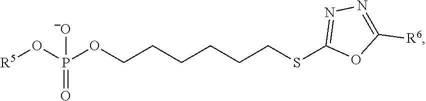

or a salt thereof.

Embodiment 83. A method of synthesizing a compound of the formula:

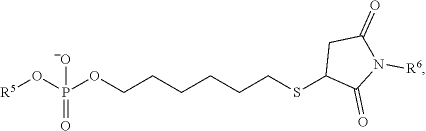

or a salt thereof, wherein:
R⁵ is a modified or unmodified oligonucleotide, or an RNAi agent, and
R⁶ is a PK/PD modulator, comprising the steps of:
(a) reacting a compound of the formula:

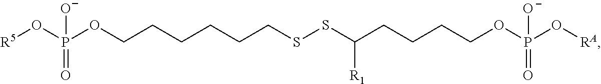

or a salt thereof
wherein R⁵ is a modified or unmodified oligonucleotide, or an RNAi agent,
R¹ is substituted or unsubstituted C₁-C₆ alkyl, and
R^{A} is a modified or unmodified nucleotide;
with a reducing agent; and
(b) adding a compound of the formula:

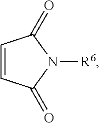

wherein R⁶ is a PK/PD modulator, to the reaction mixture.

Embodiment 84. A method of synthesizing a compound of the formula:

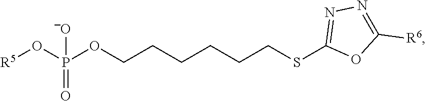

or a salt thereof, wherein:
R⁵ is a modified or unmodified oligonucleotide, or an RNAi agent, and
R⁶ is a PK/PD modulator, comprising the steps of:
(a) reacting a compound of the formula:

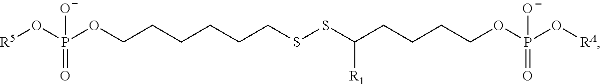

or a salt thereof wherein R⁵ is a modified or unmodified oligonucleotide,
R¹ is substituted or unsubstituted C₁-C₆ alkyl, and
R^{A} is a modified or unmodified nucleotide, or an RNAi agent;
with a reducing agent; and
(b) adding a compound of the formula:

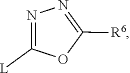

wherein L is a leaving group and R⁶ is a PK/PD modulator, to the reaction mixture.

Embodiment 85. The method of embodiment 82 or 84, wherein L is a sulfonate leaving group.

Embodiment 86. The method of any one of embodiments 82, or 84-85, wherein L is-SO₂Me.

Embodiment 87. The method of any one of embodiments 80-86, wherein the reducing agent is selected from the group consisting of tris(2-carboxyethyl) phosphine hydrochloride (TCEP), 2-mercaptoethanol, dithiothreitol (DTT), and hydroxylamine.

Embodiment 88. The method of any one of embodiments 80-87, wherein R^{A} is modified or unmodified adenosine, modified or unmodified cytidine, modified or unmodified guanosine, modified or unmodified uridine, modified or unmodified inosine, or modified or unmodified deoxythymidine.

Embodiment 89. The method of any one of embodiments 80-88, wherein R^{A} is modified or unmodified deoxythymidine.

Embodiment 90. The method of any one of embodiments 80-89, wherein R¹ is substituted or unsubstituted methyl.

Embodiment 91. The method of any one of embodiments 80-90, wherein R¹ is unsubstituted methyl.

Embodiment 92. The method of any one of embodiments 80-91, wherein R⁵ comprises one or more modified nucleotides.

Embodiment 93. The method of embodiment 92, wherein the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 94. The method of any one of embodiments 80-93, wherein R⁵ comprises one or more modified internucleoside linkages.

Embodiment 95. The method of any one of embodiments 81-94, wherein R⁶ is of the formula:

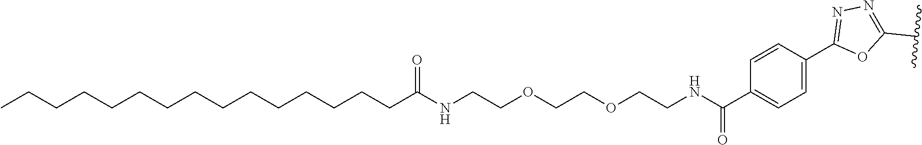 [SMILES: *c1nnc(-c2ccc(C(=O)NCCOCCOCCNC(=O)CCCCCCCCCCCCCCC)cc2)o1]

Embodiment 96. A method of synthesizing an oligonucleotide comprising the steps of
(a) coupling a first nucleotide comprising a trityl group, or a first plurality of linked nucleotides comprising a trityl group to the functionalized solid support of any one of embodiments 1-25;
(b) optionally washing the functionalized solid support after coupling of the first nucleotide or the first plurality of linked nucleotides has been coupled to the solid support with a wash comprising acetic anhydride or N-methylimidazole (NMI), and an organic solvent;
(c) detritylating the first nucleotide or the first plurality of linked nucleotides with a reagent comprising a strong acid and an organic solvent to form a detritylated nucleotide or a plurality of detritylated linked nucleotides;
(d) washing the detritylated nucleotide or the plurality of detritylated linked nucleotides with a wash comprising a second organic solvent;
(e) coupling a second nucleotide comprising a trityl group, or a second plurality of linked nucleotides comprising a trityl group, to the detritylated nucleotide or the plurality of linked detritylated nucleotides under reaction conditions that promote coupling; and
(f) repeating steps (b) through (e) one or more times, wherein the method is carried out in a single reaction vessel.

What is claimed is:

1. A functionalized solid support for oligonucleotide synthesis comprising the structure: 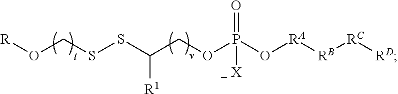
wherein R is hydrogen, a modified or unmodified nucleotide, a modified or unmodified oligonucleotide, RNAi agent, or an oxygen protecting group;
R¹ is substituted or unsubstituted C₁-C₆ alkyl;
X is O or S;
R^{A} is a modified or unmodified nucleotide;
R^{B} is a linker;
R^{C} is a spacer;
R^{D} is a solid support material
t is an integer between 1 and 20, inclusive; and
v is an integer between 1 and 20, inclusive.

2. The compound of claim 1, wherein t is 3, 4, 5, 6, 7, or 8.

3. The compound of claim 1 or 2, wherein v is 2, 3, 4, 5, or 6.

4. A functionalized solid support for oligonucleotide synthesis comprising the structure: 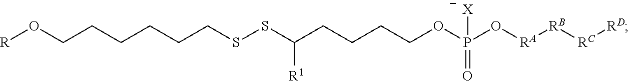
wherein R is hydrogen, a modified or unmodified nucleotide, a modified or unmodified oligonucleotide, RNAi agent, or an oxygen protecting group;
R¹ is substituted or unsubstituted C₁-C₆ alkyl;
X is O or S;
R^{A} is a modified or unmodified nucleotide;
R^{B} is a linker;
R^{C} is a spacer; and
R^{D} is a solid support material.

5. The solid support of any one of claims 1-4, wherein R^{A} is modified or unmodified adenosine nucleotide, modified or unmodified cytidine nucleotide, modified or unmodified guanosine nucleotide, modified or unmodified uridine nucleotide, modified or unmodified inosine nucleotide, or modified or unmodified deoxythymidine nucleotide.

6. The solid support of any one of claims 1-5, wherein R^{A} is modified or unmodified deoxythymidine nucleotide.

7. The solid support of any one of claims 1-6 comprising the structure: 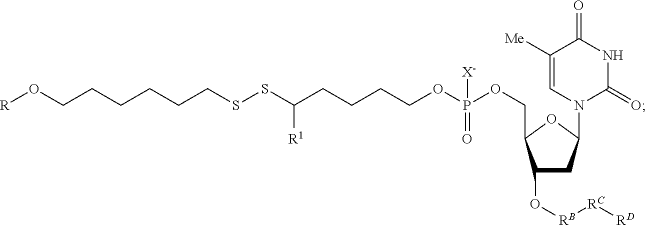
wherein R is hydrogen, a modified or unmodified nucleotide, or an oxygen protecting group;
R¹ is substituted or unsubstituted C₁-C₆ alkyl;
X is O or S;
R^{B} is a linker;
R^{C} is a spacer; and
R^{D} is a solid support material.

8. The solid support of any one of claims 1-7, wherein R^{B} is of the formula:

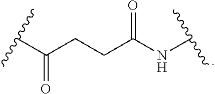 [SMILES: *NC(=O)CCC(*)=O]

9. The solid support of any one of claims 1-8, comprising the structure: 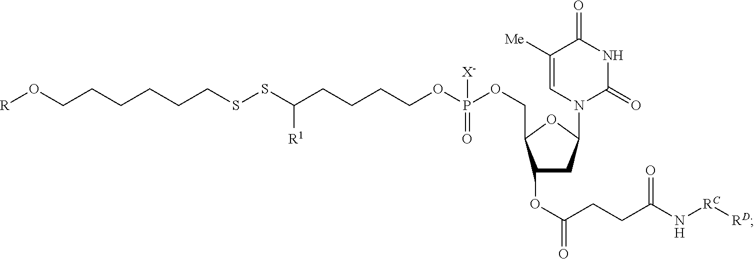
wherein R is hydrogen, a modified or unmodified nucleotide, or an oxygen protecting group;
R¹ is substituted or unsubstituted C₁-C₆ alkyl;
X is O or S;
R^{C} is a spacer; and
R^{D} is a solid support material.

10. The solid support of any one of claims 1-9, wherein R^{C} is of the formula: 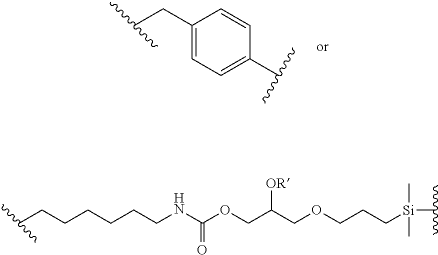
wherein R′ is C₁-C₆ alkyl.

11. The solid support of any one of claims 1-10 comprising the structure: 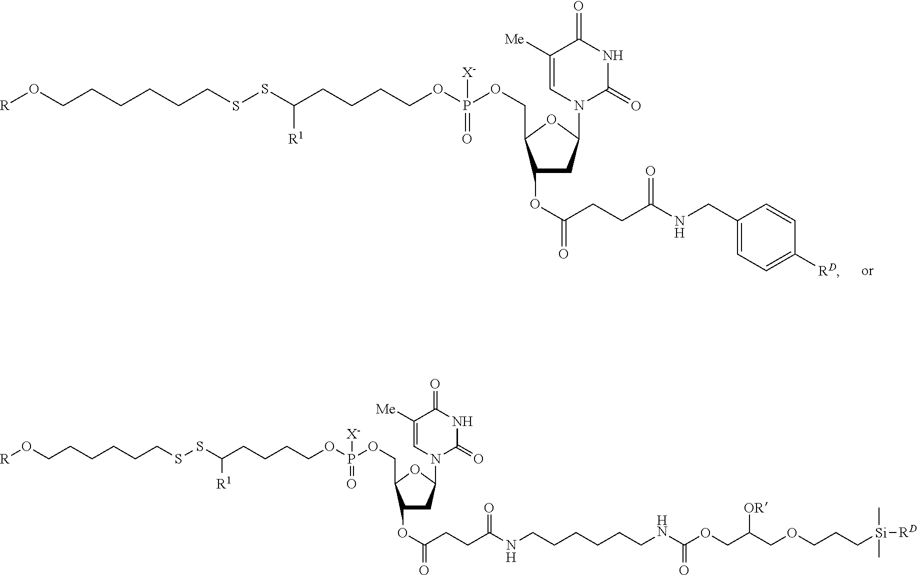
wherein R is hydrogen, a modified or unmodified nucleotide, a modified or unmodified oligonucleotide, RNAi agent, or an oxygen protecting group;
X is O or S;
R¹ is substituted or unsubstituted C₁-C₆ alkyl;
R′ is C₁-C₆ alkyl; and
R^{D} is a solid support material.

12. The solid support of any one of claims 1-11, wherein the solid support material comprises polystyrene or controlled pore glass (CPG).

13. The solid support of any one of claims 1-12, wherein R is an oxygen protecting group.

14. The solid support of any one of claims 1-13, wherein R is a dimethoxytrityl (DMT) protecting group.

15. The solid support of any one of claims 1-12, wherein R is a modified or unmodified nucleotide.

16. The solid support of claim 15, wherein the oligonucleotide comprises one or more modified nucleotides.

17. The solid support of claim 16, wherein the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

18. The solid support of any one of claims 15-17, wherein the oligonucleotide comprises one or more modified internucleoside linkages.

19. The solid support of any one of claims 15-18, wherein the oligonucleotide comprises a sequence that is between 18 and 30 nucleotides in length.

20. The solid support of claim 19, wherein the oligonucleotide comprises a sequence that is between 18 and 24 nucleotides in length.

21. The solid support of claim 19 or 20, wherein the oligonucleotide comprises a sequence that is 21 nucleotides in length.

22. The solid support of any one of claims 1-21, wherein R¹ is substituted or unsubstituted methyl.

23. The solid support of any one of claims 1-22, wherein R¹ is unsubstituted methyl.

24. The solid support of any one of claims 1-23, wherein X is O.

25. The solid support of any one of claims 1-24, wherein X is S.

26. A compound of the Formula (III′): 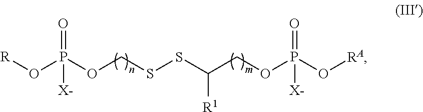
or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S;
R¹ is substituted or unsubstituted C₁-C₆ alkyl;
n is an integer between 0 and 20, inclusive;
and m is an integer between 0 and 20, inclusive.

27. The compound of claim 26, wherein n is 6.

28. The compound of claim 26 or 27, wherein m is 4.

29. A compound of the Formula (III): 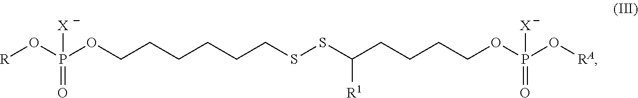
or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S; and
R¹ is substituted or unsubstituted C₁-C₆ alkyl.

30. A compound of the formula (IV′): 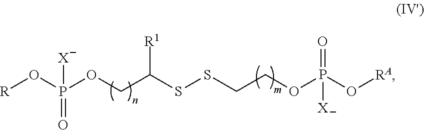
or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S;
R¹ is substituted or unsubstituted C₁-C₆ alkyl;
n is an integer between 0 and 20, inclusive;
and m is an integer between 0 and 20, inclusive.

31. The compound of claim 30, wherein n is 4.

32. The compound of claim 30 or 31, wherein m is 5.

33. A compound of the formula (IV): 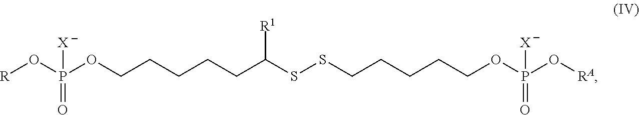
or a salt thereof, wherein:
R is hydrogen, a modified or unmodified nucleotide, an RNAi agent, or a modified or unmodified oligonucleotide;
R^{A} is a modified or unmodified nucleotide;
each X is independently O or S; and
R¹ is substituted or unsubstituted C₁-C₆ alkyl.

34. The compound of any one of claims 26-33, wherein at least one instance of X is S.

35. The compound of any one of claims 26-33, wherein at least one instance of X is O.

36. The compound of any one of claims 26-35, wherein R^{A} is a modified or unmodified nucleotide.

37. The compound of any one of claims 26-36, wherein R^{A} is modified or unmodified adenosine, modified or unmodified cytidine, modified or unmodified guanosine, modified or unmodified uridine, modified or unmodified inosine, or modified or unmodified thymidine.

38. The compound of any one of claims 26-37, wherein R^{A} is modified or unmodified thymidine.

39. The compound of any one of claims 26-38, wherein R¹ is substituted or unsubstituted methyl.

40. The compound of any one of claims 26-39, wherein R¹ is unsubstituted methyl.

41. The compound of any one of claim 26-29, or 34-40, of the formula: 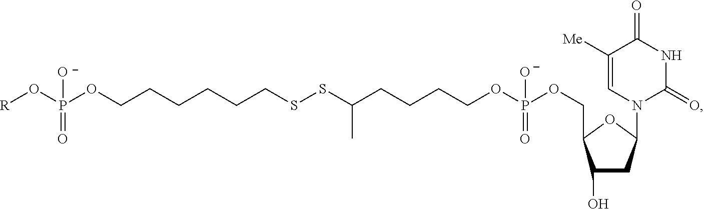
or a salt thereof.

42. The compound of any one of claims 30-40, of the formula: 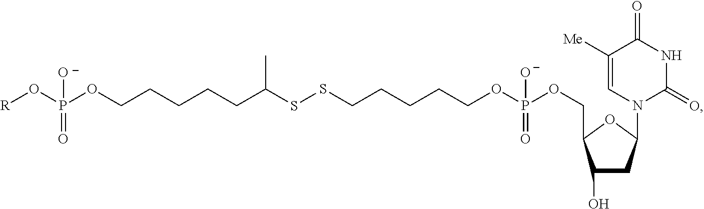
or a salt thereof.

43. The compound of any one of claims 26-42, wherein R is a modified or unmodified oligonucleotide.

44. The compound of claim 43, wherein the oligonucleotide comprises one or more modified nucleotides.

45. The compound of claim 44, wherein the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

46. The compound of any one of claims 43-45, wherein the oligonucleotide comprises one or more modified internucleoside linkages.

47. The compound of any one of claims 43-46, wherein the oligonucleotide comprises a sequence that is between 18 and 30 nucleotides in length.

48. The compound of claim 47, wherein the oligonucleotide comprises a sequence that is between 18 and 24 nucleotides in length.

49. The compound of claim 47 or 48, wherein the oligonucleotide comprises a sequence that is 21 nucleotides in length.

50. A compound of the formula (I′): 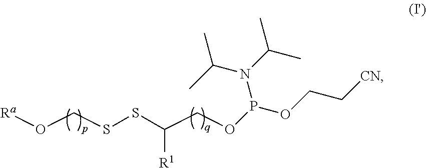
or a salt thereof, wherein:
R^{a} is hydrogen, a modified or unmodified nucleotide, an RNAi agent, or an oxygen protecting group;
R¹ is substituted or unsubstituted C₁-C₆ alkyl;
p is an integer between 0 and 20, inclusive; and
q is an integer between 0 and 20, inclusive.

51. The compound of claim 50, wherein p is 6.

52. The compound of claim 50 or 51, wherein q is 4.

53. A compound of the formula (I): 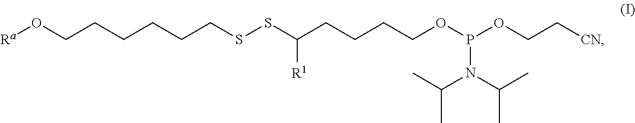
or a salt thereof, wherein:
R^{a} is hydrogen, a modified or unmodified nucleotide, an RNAi agent, or an oxygen protecting group; and R¹ is substituted or unsubstituted C₁-C₆ alkyl.

54. A compound of the formula (V′): 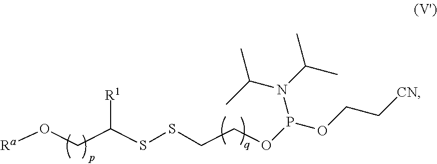
or a salt thereof, wherein:
R^{a} is hydrogen, a modified or unmodified nucleotide, an RNAi agent, or an oxygen protecting group;
R¹ is substituted or unsubstituted C₁-C₆ alkyl;
p is an integer between 0 and 20, inclusive; and
q is an integer between 0 and 20, inclusive.

55. The compound of claim 54, wherein p is 4.

56. The compound of claim 54 or 55, wherein q is 5.

57. A compound of the formula (V): 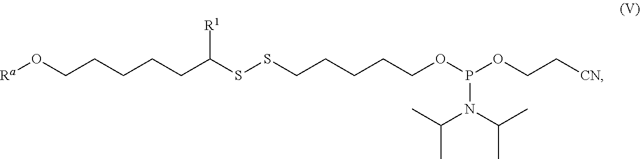
or a salt thereof, wherein:
R^{a} is hydrogen, a modified or unmodified nucleotide, an RNAi agent, or an oxygen protecting group; and R¹ is substituted or unsubstituted C₁-C₆ alkyl.

58. The compound of any one of claims 50-57, wherein R^{a} is an oxygen protecting group.

59. The compound of any one of claims 50-58, wherein R^{a} is a dimethoxytrityl (DMT) protecting group.

60. The compound of any one of claim 50-53, or 58-59, of the formula: 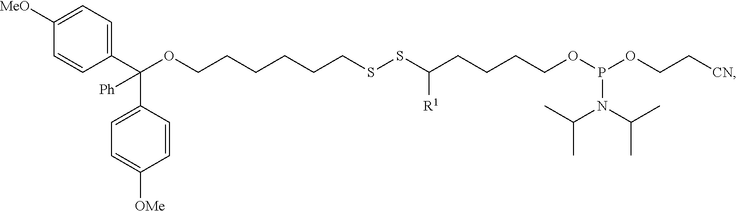
or a salt thereof wherein R¹ is substituted or unsubstituted C₁-C₆ alkyl.

61. The compound of any one of claims 54-59, of the formula: 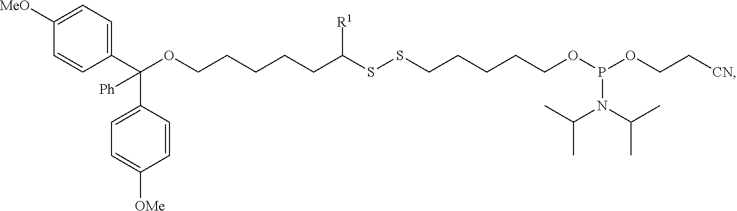
or a salt thereof wherein R¹ is substituted or unsubstituted C₁-C₆ alkyl.

62. The compound of any one of claims 50-61, wherein R¹ is substituted or unsubstituted methyl.

63. The compound of any one of claims 50-62, wherein R¹ is unsubstituted methyl.

64. The compound of any one of claim 50-53, 58-60, or 62-63, of the formula: 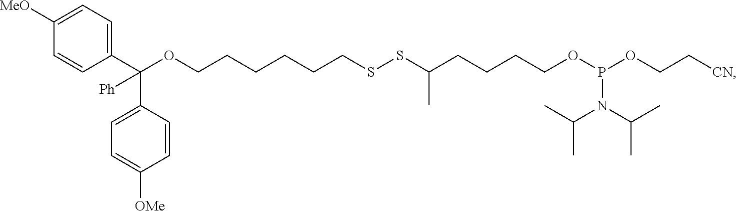 [SMILES: COc1ccc(C(OCCCCCCSSC(C)CCCCOP(OCCC#N)N(C(C)C)C(C)C)(c2ccccc2)c2ccc(OC)cc2)cc1]
or a salt thereof.

65. The compound of any one of claim 54-59, or 61-63, of the formula: 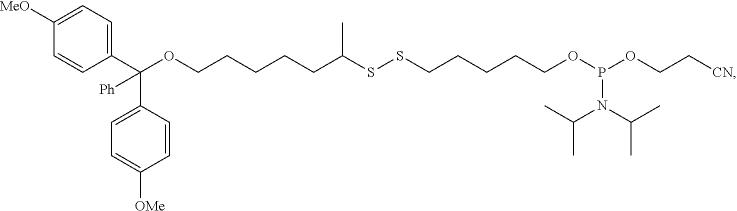 [SMILES: COc1ccc(C(OCCCCCC(C)SSCCCCCOP(OCCC#N)N(C(C)C)C(C)C)(c2ccccc2)c2ccc(OC)cc2)cc1]
or a salt thereof.

66. A compound of Formula (II): 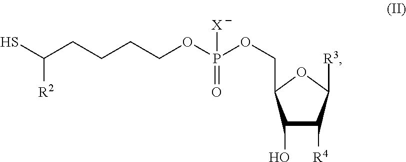
or a salt thereof, wherein:
R² is substituted or unsubstituted C₁-C₆ alkyl;
X is O or S;
R³ is a nucleobase; and
R⁴ is hydrogen, methoxy, or a halogen.

67. The compound of claim 66, wherein R⁴ is hydrogen.

68. The compound of claim 66 or 67, wherein R³ is cytosine, guanine, adenine, thymine, or uracil.

69. The compound of any one of claims 66-68, wherein R³ is thymine.

70. The compound of any one of claims 66-69, wherein R² is substituted or unsubstituted methyl.

71. The compound of any one of claims 66-70, wherein R² is unsubstituted methyl.

72. The compound of any one of claims 66-71, of the formula: 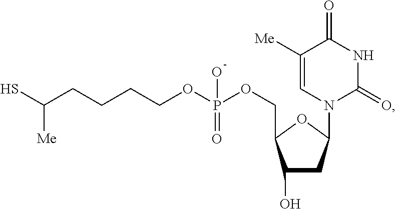
or a salt thereof.

73. A method comprising the step of reacting a support bound oligonucleotide comprising the structure: 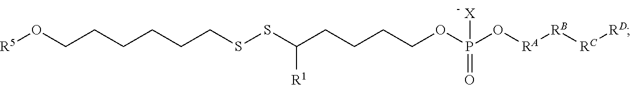
wherein R⁵ is a modified or unmodified oligonucleotide, or an RNAi agent;
R¹ is substituted or unsubstituted C₁-C₆ alkyl;
R^{A} is a modified or unmodified nucleotide;
R^{B} is a linker;
R^{C} is a spacer; and
R^{D} is a solid support material;
with an organic base to form an oligonucleotide of the formula: 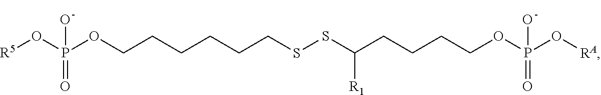
or a salt thereof.

74. The method of claim 73, wherein R^{B} is of the formula:

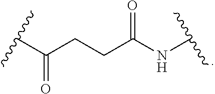 [SMILES: *NC(=O)CCC(*)=O]

75. The method of claim 73 or 74, wherein R^{C} is of the formula: 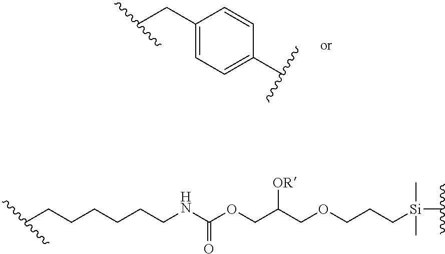
wherein R′ is C₁-C₆ alkyl.

76. The method of any one of claims 73-75, wherein the solid support material comprises polystyrene or controlled pore glass (CPG).

77. The method of any one of claims 73-76, wherein the organic base is an amine base.

78. The method of any one of claims 73-77, wherein the organic base is an alkylamine.

79. The method of any one of claims 73-78, wherein the organic base is methylamine.

80. A method comprising the steps of reacting a compound of the formula: 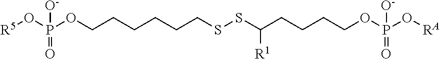
wherein R⁵ is a modified or unmodified oligonucleotide,
R¹ is substituted or unsubstituted C₁-C₆ alkyl, and
R^{A} is a modified or unmodified nucleotide, or an RNAi agent;
with a reducing agent to form a compound of the formula: 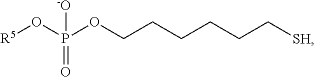
or a salt thereof.

81. The method of claim 80, further comprising the step of reacting the compound of the formula: 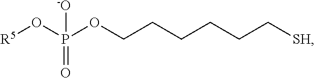
or a salt thereof wherein R⁵ is a modified or unmodified oligonucleotide, or an RNAi agent, with a compound of the formula: 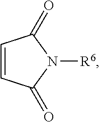
wherein R⁶ is a PK/PD modulator, to form a compound of the formula: 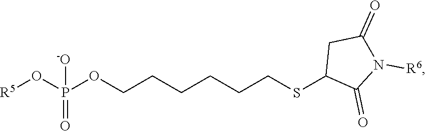
or a salt thereof.

82. The method of claim 80, further comprising the step of reacting the compound of the formula: 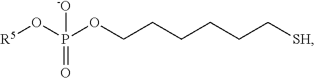
or a salt thereof, wherein R⁵ is a modified or unmodified oligonucleotide with a compound of the formula: 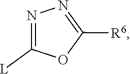
wherein:
R⁶ is a PK/PD modulator, and
L is a leaving group;
to form a compound of the formula: 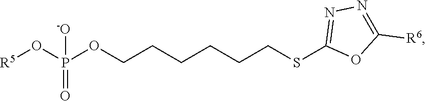
or a salt thereof.

83. A method of synthesizing a compound of the formula: 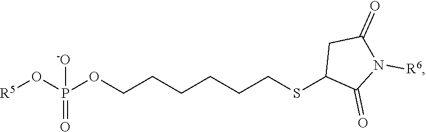
or a salt thereof, wherein:
R⁵ is a modified or unmodified oligonucleotide, or an RNAi agent, and
R⁶ is a PK/PD modulator, comprising the steps of:
(a) reacting a compound of the formula: 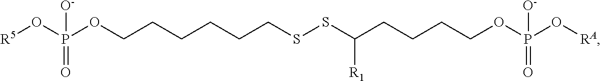
or a salt thereof wherein R⁵ is a modified or unmodified oligonucleotide, or an RNAi agent,
R¹ is substituted or unsubstituted C₁-C₆ alkyl, and
R^{A} is a modified or unmodified nucleotide;
with a reducing agent; and
(b) adding a compound of the formula: 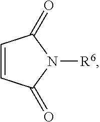
wherein R⁶ is a PK/PD modulator, to the reaction mixture.

84. A method of synthesizing a compound of the formula: 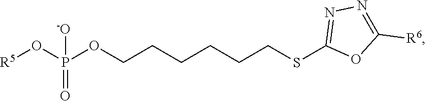
or a salt thereof, wherein:
R⁵ is a modified or unmodified oligonucleotide, or an RNAi agent, and
R⁶ is a PK/PD modulator, comprising the steps of:
(a) reacting a compound of the formula: 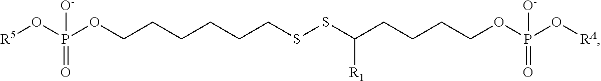
or a salt thereof
wherein R⁵ is a modified or unmodified oligonucleotide,
R¹ is substituted or unsubstituted C₁-C₆ alkyl, and
R^{A} is a modified or unmodified nucleotide, or an RNAi agent;
with a reducing agent; and
(b) adding a compound of the formula: 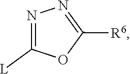
wherein L is a leaving group and R⁶ is a PK/PD modulator, to the reaction mixture.

85. The method of claim 82 or 84, wherein L is a sulfonate leaving group.

86. The method of any one of claim 82, or 84-85, wherein L is —SO₂Me.

87. The method of any one of claims 80-86, wherein the reducing agent is selected from the group consisting of tris(2-carboxyethyl) phosphine hydrochloride (TCEP), 2-mercaptoethanol, dithiothreitol (DTT), and hydroxylamine.

88. The method of any one of claims 80-87, wherein R^{A} is modified or unmodified adenosine, modified or unmodified cytidine, modified or unmodified guanosine, modified or unmodified uridine, modified or unmodified inosine, or modified or unmodified deoxythymidine.

89. The method of any one of claims 80-88, wherein R^{A} is modified or unmodified deoxythymidine.

90. The method of any one of claims 80-89, wherein R¹ is substituted or unsubstituted methyl.

91. The method of any one of claims 80-90, wherein R¹ is unsubstituted methyl.

92. The method of any one of claims 80-91, wherein R⁵ comprises one or more modified nucleotides.

93. The method of claim 92, wherein the one or more modified nucleotides are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

94. The method of any one of claims 80-93, wherein R⁵ comprises one or more modified internucleoside linkages.

95. The method of any one of claims 81-94, wherein R⁶ is of the formula:

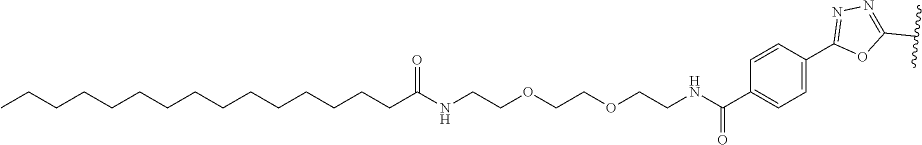 [SMILES: *c1nnc(-c2ccc(C(=O)NCCOCCOCCNC(=O)CCCCCCCCCCCCCCC)cc2)o1]

96. A method of synthesizing an oligonucleotide comprising the steps of
(a) coupling a first nucleotide comprising a trityl group, or a first plurality of linked nucleotides comprising a trityl group to the functionalized solid support of any one of claims 1-25;
(b) optionally washing the functionalized solid support after coupling of the first nucleotide or the first plurality of linked nucleotides has been coupled to the solid support with a wash comprising acetic anhydride or N-methylimidazole (NMI), and an organic solvent;
(c) detritylating the first nucleotide or the first plurality of linked nucleotides with a reagent comprising a strong acid and an organic solvent to form a detritylated nucleotide or a plurality of detritylated linked nucleotides;
(d) washing the detritylated nucleotide or the plurality of detritylated linked nucleotides with a wash comprising a second organic solvent;
(e) coupling a second nucleotide comprising a trityl group, or a second plurality of linked nucleotides comprising a trityl group, to the detritylated nucleotide or the plurality of linked detritylated nucleotides under reaction conditions that promote coupling; and
(f) repeating steps (b) through (e) one or more times,
wherein the method is carried out in a single reaction vessel.


SEQUENCE LISTING (7 sequences)

SEQ ID NO: 1 - RNA, 22 nt, synthetic construct
  modified_base 1: mod_base = gm
  modified_base 2: mod_base = gm
  modified_base 3: mod_base = um
  modified_base 4: mod_base = cm
  modified_base 5: mod_base = OTHER; note = 2'-O-methyladenosine
  modified_base 6: mod_base = um
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methyladenosine
  modified_base 21^22: mod_base = OTHER; note = phosphorothioate linkage
  modified_base 22: mod_base = OTHER; note = 2'-deoxythymidine
  source 1..22: mol_type = other RNA
ggtcatgatcttgctgtaacat

SEQ ID NO: 2 - RNA, 22 nt, synthetic construct
  modified_base 1: mod_base = gm
  modified_base 2: mod_base = gm
  modified_base 3: mod_base = um
  modified_base 4: mod_base = cm
  modified_base 5: mod_base = OTHER; note = 2'-O-methyladenosine
  modified_base 6: mod_base = um
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methyladenosine
  modified_base 21^22: mod_base = OTHER; note = phosphorothioate linkage
  modified_base 22: mod_base = OTHER; note = 2'-deoxythymidine
  source 1..22: mol_type = other RNA
ggtcatgatcttgctgtaacat

SEQ ID NO: 3 - RNA, 22 nt, synthetic construct
  modified_base 1: mod_base = cm
  modified_base 1^2: mod_base = OTHER; note = phosphorothioate linkage
  modified_base 2: mod_base = cm
  modified_base 3: mod_base = um
  modified_base 4: mod_base = gm
  modified_base 5: mod_base = um
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = um
  modified_base 21: mod_base = OTHER; note = 2'-O-methyladenosine
  modified_base 21^22: mod_base = OTHER; note = phosphorothioate linkage
  modified_base 22: mod_base = OTHER; note = 2'-deoxythymidine
  source 1..22: mol_type = other RNA
cctgtgcaaccagaacaaatat

SEQ ID NO: 4 - RNA, 22 nt, synthetic construct
  modified_base 1: mod_base = cm
  modified_base 1^2: mod_base = OTHER; note = phosphorothioate linkage
  modified_base 2: mod_base = cm
  modified_base 3: mod_base = um
  modified_base 4: mod_base = gm
  modified_base 5: mod_base = um
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = um
  modified_base 21: mod_base = OTHER; note = 2'-O-methyladenosine
  modified_base 21^22: mod_base = OTHER; note = phosphorothioate linkage
  modified_base 22: mod_base = OTHER; note = 2'-deoxythymidine
  source 1..22: mol_type = other RNA
cctgtgcaaccagaacaaatat

SEQ ID NO: 5 - RNA, 22 nt, synthetic construct
  modified_base 1: mod_base = cm
  modified_base 1^2: mod_base = OTHER; note = phosphorothioate linkage
  modified_base 2: mod_base = cm
  modified_base 3: mod_base = um
  modified_base 4: mod_base = gm
  modified_base 5: mod_base = um
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = um
  modified_base 21: mod_base = OTHER; note = 2'-O-methyladenosine
  modified_base 21^22: mod_base = OTHER; note = phosphorothioate linkage
  modified_base 22: mod_base = OTHER; note = 2'-deoxythymidine
  source 1..22: mol_type = other RNA
cctgtgcaaccagaacaaatat

SEQ ID NO: 6 - RNA, 22 nt, synthetic construct
  modified_base 1: mod_base = cm
  modified_base 2: mod_base = OTHER; note = 2'-O-methyladenosine
  modified_base 3: mod_base = gm
  modified_base 4: mod_base = gm
  modified_base 5: mod_base = OTHER; note = 2'-O-methyladenosine
  modified_base 6: mod_base = um
  modified_base 7: mod_base = um
  modified_base 8: mod_base = cm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = cm
  modified_base 14: mod_base = um
  modified_base 15: mod_base = gm
  modified_base 16: mod_base = gm
  modified_base 17: mod_base = um
  modified_base 18: mod_base = um
  modified_base 19: mod_base = um
  modified_base 20: mod_base = cm
  modified_base 21: mod_base = OTHER; note = 2'-O-methyladenosine
  modified_base 21^22: mod_base = OTHER; note = phosphorothioate linkage
  modified_base 22: mod_base = OTHER; note = 2'-deoxythymidine
  source 1..22: mol_type = other RNA
caggattcagatctggtttcat

SEQ ID NO: 7 - RNA, 22 nt, synthetic construct
  modified_base 1: mod_base = cm
  modified_base 2: mod_base = OTHER; note = 2'-O-methyladenosine
  modified_base 3: mod_base = gm
  modified_base 4: mod_base = gm
  modified_base 5: mod_base = OTHER; note = 2'-O-methyladenosine
  modified_base 6: mod_base = um
  modified_base 7: mod_base = um
  modified_base 8: mod_base = cm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = cm
  modified_base 14: mod_base = um
  modified_base 15: mod_base = gm
  modified_base 16: mod_base = gm
  modified_base 17: mod_base = um
  modified_base 18: mod_base = um
  modified_base 19: mod_base = um
  modified_base 20: mod_base = cm
  modified_base 21: mod_base = OTHER; note = 2'-O-methyladenosine
  modified_base 21^22: mod_base = OTHER; note = phosphorothioate linkage
  modified_base 22: mod_base = OTHER; note = 2'-deoxythymidine
  source 1..22: mol_type = other RNA
caggattcagatctggtttcat